(19) United States
(12) Patent Application Publication
(10) Pub. No.: US 2026/0144619 A1
(43) Pub. Date: May 28, 2026

(54) DEVICES, SYSTEMS, AND METHODS RELATING TO A WIRELESSLY ACTUATED MICROFLUIDIC PUMP AND VALVE FOR CONTROLLED LIQUID DELIVERY IN DENTAL IMPLANTS

(71) Applicant: VANDERBILT UNIVERSITY, Nashville, TN (US)

(72) Inventor: Xiaoguang DONG, Nashville, TN (US)

(21) Appl. No.: 19/402,096

(22) Filed: Nov. 26, 2025

Related U.S. Application Data
(60) Provisional application No. 63/725,976, filed on Nov. 27, 2024.

(51) Int. Cl. A61C 8/02; A61C 8/00; A61M 31/00

(52) U.S. Cl. CPC A61C 8/0006; A61C 8/0022; A61M 31/002; A61M 2205/0272; A61M 2205/04; A61M 2205/3334; A61M 2210/0631

(57) ABSTRACT

Described herein are devices, systems, and methods relating to dental implants, in particular dental implants that can store and/or deliver medicaments to a subject. In embodiments, devices, systems, and methods as described herein can be utilized to deliver medicaments to the bone-implant interface in a subject.

### CROSS-REFERENCE TO RELATED APPLICATION[S]

This application claims priority to, and the benefit of, U.S. Provisional Patent Application Ser. No. 63/725,976, entitled “DEVICES, SYSTEMS, AND METHODS RELATING TO A WIRELESSLY ACTUATED MICROFLUIDIC PUMP AND VALVE FOR CONTROLLED LIQUID DELIVERY IN DENTAL IMPLANTS” and filed on Nov. 27, 2024, the entire contents of which are incorporated herein by reference as if set forth in its entirety.

### BACKGROUND

Enabling minimally invasive and precise control of liquid release in dental implants is crucial for therapeutic functions such as delivering antibiotics to prevent biofilm formation, infusing stem cells to promote osseointegration, and administering other biomedicines. However, achieving controllable liquid cargo release in dental implants remains challenging due to the lack of wireless and miniaturized fluidic control mechanisms. Accordingly, there is a need to address the aforementioned deficiencies and inadequacies.

### SUMMARY

Described herein are dental implants, systems, and methods of use. In embodiments, described herein is a dental implant. Dental implants according to the present disclosure can comprise an internal fluidic chamber extending axially through the implant. Dental implants according to the present disclosure can comprise one or more inner microfluidic channels extending radially from the internal fluidic chamber to one or more outer microfluidic channels. Dental implants according to the present disclosure can comprise one or more outer microfluidic channels. In embodiments, the outer microfluidic channels comprise a helical groove transversing outer threads of the implant; an actuator configured within the internal fluidic chamber to pump liquid through the implant in the presence of a magnetic field. Dental implants according to the present disclosure can comprise a flexible magnetic valve configured between the internal fluidic chamber and the one or more inner microfluidic channels. In embodiments, the flexible magnetic valve can be opened or closed through application of an axial external magnetic field. Dental implants according to the present disclosure can comprise a stopper structure bonded to the flexible magnetic valve. In embodiments, the stopper structure can maintain an opening angle or a closed position of the flexible magnetic valve after the axial external magnetic field has been removed.

In embodiments, the actuator can comprise a magnet-screw piston. In embodiments, the actuator can comprise a screw bonded to a magnet, wherein an epoxy bonds the screw to the magnet, and a biocompatible coating. In embodiments, the screw can comprise stainless steel, titanium, or both. In embodiments, the magnet can be a NdFeB magnet. In embodiments, the coating can comprise PDMS, parylene-C, or both. In embodiments, the stopper structure can be coated with parylene-C. In embodiments, the internal fluidic chamber can comprise a small inlet, wherein the internal fluidic chamber can be refilled with the liquid. In embodiments, implant can be titanium. In embodiments, the flexible magnetic valve can be bonded to the stopper structure using uncured PDMS.

In embodiments, described herein are systems. In embodiments, a system for delivering a liquid cargo within a dental implant can comprise a dental implant as described herein, and a magnetic actuation unit. In embodiments, a magnetic actuation unit can comprise at least one or more of a magnet. In embodiments, the magnet can be mounted onto a step motor. In embodiments, the step motor can be controlled by a step motor driver; Magnetic actuation units according to the present disclosure can comprise a battery. In embodiments, the battery can be connected to the step motor driver and a microcontroller. In embodiments, the microcontroller can control the speed of the step motor driver. Magnetic actuation units according to the present disclosure can comprise a switch. In embodiments, the switch can be connected to the microcontroller and can allow a user to modify the speed of the step motor driver. In embodiments, the magnetic actuation unit can generate the external magnetic field, thereby controlling delivery of the liquid through the implant. In embodiments, the actuator comprises a stainless steel or titanium screw bonded to a NdFeB magnet, wherein an epoxy bonds the screw to the magnet; and a biocompatible coating, wherein the coating further comprises PDMS and parylene-C. In embodiments, the stopper structure can be coated with parylene-C. In embodiments, the internal fluidic chamber can comprise a small inlet. In embodiments, the internal fluidic chamber can be refilled with the liquid. In embodiments, the implant can be titanium. In embodiments, the flexible magnetic valve can be bonded to the stopper structure using uncured PDMS.

Also described herein are methods of using dental implants and systems as described herein. In embodiments, described herein are methods of introducing a medicament to an implant-bone interface. In embodiments, methods can comprise implanting a dental implant as described herein into the jawbone of a patient. In embodiments, the implant contains a medicament (for example, antibiotic, bioagent, coating, or stem cells). In embodiments, methods can comprise opening a flexible magnetic valve within the implant. In embodiments, the flexible magnetic valve can be opened by application of an axial external magnetic field; activating an actuator within the implant, wherein the actuator pumps the medicament through the flexible magnetic valve and out of the implant by application of a rotating external magnetic field; and delivering the medicament from the implant directly to the implant-bone interface. In embodiments, the dental implant can be refilled by injecting the medicament into a small inlet of the implant. In embodiments, the medicament can comprise one or more antibiotics, stem cells, coatings, small molecules less than 2500 daltons, or a combination of any thereof. In embodiments, the actuator can be further configured to translate along the axis of the internal fluidic chamber when the rotating magnetic field is applied. In embodiments, the actuator can be further configured to translate along the axis of the internal fluidic chamber when the rotating magnetic field is applied by the magnetic actuation unit.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1D: Concept of a wirelessly controlled dental implant for on-demand liquid therapeutic agent delivery. FIG. 1A shows a concept of a dental implant for on-demand release of therapeutic agents in a liquid form. The pump and valve are controlled by external magnetic fields. Created by Biorender.com. FIG. 1B shows optical images of the dental implant set. The set includes a dental screw, a magnetic valve, and a magnetic pump with a magnet-screw piston to be placed inside a dental screw. FIG. 1C shows an illustration of the proposed wirelessly actuated pump and valve mechanisms inside a dental implant. A magnet-screw piston is wirelessly actuated to pump liquid cargos from the internal channel to the surface of the dental implant via the outer channels. A flexible magnetic valve is integrated to control the opening or closing. FIG. 1D shows optical images of a dental implant inside a phantom before and after ejecting liquids from the internal fluidic chamber to the phantom-implant interface.

FIGS. 2A-2G: Design and fabrication of a dental implant integrating fluidic channels, pumps, and valves. FIG. 2A is an illustration of a dental implant with fluidic modules after assembly. FIG. 2B is an illustration of the magnet-screw piston pump and the assembly process. The magnet-screw piston is created by bonding a commercial screw (M2 by 2 mm) to a cube NdFeB magnet (1 mm by 1 mm by 1 mm). The magnet and screw are further coated with PDMS and parylene-C for biocompatibility. FIG. 2C is an illustration of the assembly process of the magnetic valve for the microfluidic channel. The stopper structure is used for maintaining the opening angle even when removing the external magnetic field. Red arrow-magnetic moment. FIG. 2D is an illustration of the fabrication process of a dental screw with fluidic channels. FIG. 2E is an optical image of a magnet-screw piston with coated PDMS and parylene-C. FIG. 2F is an optical image of a modular pump with a magnet-screw piston inside. FIG. 2G is an optical image of a dental screw made of resin by a transfer molding method.

FIGS. 3A-3E: Characterization of the liquid pumping performance of a magnet-screw piston in a dental implant. FIG. 3A is an illustration of the fluid pumping mechanism of a magnetically actuated piston pump. Red arrow: the magnetic moment of the magnet-screw piston. Blue arrow: magnetic field in the x-y plane B_{xy}. The magnetic torque is given by τₘ=Mₚ×B_{xy}. FIG. 3B shows video frames of the dental implant pumping DI water mixed with blue dye. A rotating magnetic field is applied with B=10 mT, f=0.1 Hz. Scale bar: 2 mm. FIG. 3C shows the traveled distance of the magnet-screw piston as a function of time when the magnet-screw piston is actuated by external magnetic fields. FIG. 3D shows an example external rotating magnetic field as a function of time allowing continuous liquid ejection. A cube NdFeB magnet (25 mm by 25 mm by 25 mm) is applied at ≈4 cm away from the magnet-screw piston. FIG. 3E shows the moving speed of the piston v divided by the actuation frequency f when varying the external magnetic field frequency and magnitude. When the magnetic field has a relatively small magnitude, the friction prevents the piston from rotating at the same frequency of the external magnetic field.

FIGS. 4A-4H: Characterization of the lockable flexible magnetic valve for controlling liquid ejection. FIG. 4A shows an illustration of the magnetic valve being closed or opened inside a dental screw. FIG. 4B shows an illustration of the mechanism of a lockable flexible magnetic valve controlled by an external magnetic field for opening and closing the fluidic channel. FIG. 4C shows video frames of the flexible magnetic valve under different magnetic fields in both the opening and closing processes. FIG. 4D shows the external magnetic field employed in FIG. 4C as a function of time. The magnitudes of the magnetic fields to open and close the valve are 60.4 mT and 40.8 mT, respectively. FIG. 4E shows the opening angle of the flexible magnetic valve as a function of time. FIG. 4F shows video frames of a modular pump with valve control. FIG. 4G shows video frames of a 3D printed dental implant with valve control. Scale bar: 5 mm. FIG. 4H shows the volume of the ejected liquid as a function of time for the dental implant in FIG. 4G.

FIGS. 5A-5I: Characterization of the liquid ejection performance in phantoms. FIG. 5A shows video frames of liquid ejection in a 3D printed dental implant placed in a phantom. g indicates the gravity direction. Scale bar: 3 mm. FIG. 4B is an illustration of the helical groove pattern on the dental implant surface. The groove pattern is a single helix. The gap size between the phantom and the dental implant is defined as the radius difference δr. FIG. 4C shows the coverage ratio of the ejected liquid when using dental implants with different gap sizes. βr/r=1%, 5%, 10%. Groove depth, 0.43 mm. FIG. 4D shows the variance of the distributed fluorescent liquids in FIG. 4C. The variance is defined as the long-axis diameter a of the fitted ellipse over the wetted area. FIG. 4E shows fluorescence images (four faces) of the ejected liquids using molded dental implants of different gap sizes. δr/r=1%, 5%, 10%. Groove depth, 0.43 mm. FIG. 4F is an illustration of the groove pattern design on the dental implant. d_{g} indicates the groove depth (width w_{g}=0.4 mm). FIG. 4G shows the coverage ratio for a dental implant with groove patterns of different depths inside a phantom. d_{g}=0.13 mm, 0.43 mm, 0.63 mm. δr/r=5%. FIG. 4H shows the variance of the distributed fluorescence liquid in FIG. 4G. FIG. 4I shows fluorescence images (four faces) of the ejected liquids using the molded dental implants with different groove pattern depths. δr/r=5%. In all experiments, B=15 mT, f=0.1 Hz. (FIGS. 4C, 4D, 4G, 4H) Liquid ejection of n=5 trials are performed for each dental screw inside a specific phantom. The circles indicate the average values for the coverage ratios or the variance of the fitted ellipse of n=20 segmented images in total for the four faces in five trials.

FIGS. 6A-6G: Demonstration of liquid ejection of a metal dental implant inside a porcine jawbone. FIG. 6A shows 3D rendering and optical images of a micromachined titanium dental screw. FIG. 6B shows optical images of a modular pump with a magnetic piston and valve. FIG. 6C shows images of a metal dental implant inside a phantom before and after releasing the liquid mixed with fluorescent dye. External magnetic field, B=15 mT and f=0.1 Hz. Scale bar: 2 mm. FIG. 6D shows optical images of the metal dental implant inside a porcine upper jawbone. FIG. 6E shows processed X-ray images of the metal dental implant inside a porcine jawbone before and after releasing the contrast agent (Omnipaque iohexol injection, GE Healthcare). The color indicates the X-ray image density (low intensity corresponds to red). Scale bar: 5 mm. FIG. 6F shows the extracted histogram of the X-ray image intensity in the periodontal area before contrast agent ejection. FIG. 6G shows an extracted histogram of the X-ray image intensity in the periodontal area after contrast agent ejection. FIG. 6E shows the intensity of the X-ray images are registered based on the intensities of the dental screws in both images.

FIGS. 7A-7D: Design of the handheld magnetic actuation system. FIG. 7A shows an optical image of the handheld magnetic actuation system. FIG. 7B shows an illustration of the magnetic actuation when the valve is closed. FIG. 7C shows an illustration of opening the valve by the handheld magnetic actuation unit. FIG. 7D shows magnetic field strength as a function of the actuation distance for cube magnets of different sizes.

FIGS. 8A-8B: Dimension of the dental implant and the fabrication methods used. FIG. 8A is an optical image and illustration of an example metal implant. FIG. 8B shows an illustration of the fabrication methods and the parameters.

FIGS. 9A-9B: Experimental setup for testing the pumping with fluorescent dye. FIG. 9A is an illustration of the experimental setup for pumping and imaging the liquid. FIG. 9B is an optical image of the experimental setup with marked components.

FIG. 10: Video frames of five trials of releasing liquids in a dental implant. The gap ratio is δr/r=5% and the groove depth is d_{g}=0.43 mm.

FIGS. 11A-11C: Optical images of the metal dental implant. FIG. 11A shows three views of the metal implant with microfluidics. FIG. 11B shows a top view of the dental implant. FIG. 11C shows a bottom view of the dental implant. Scale bars, 2 mm.

FIG. 12: Demonstration of sealing using the flexible magnetic valve inside a modular pump and a metal screw.

FIG. 13: Additional aspects of an embodiment of an implant according to the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of genetics, microbiology, biochemistry, molecular biology, cellular biology, tissue culture, therapeutic administrations and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature.

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

The terms “administering,” “delivering,” and “introducing,” can be used interchangeably to indicate the introduction of a therapeutic composition or agent (e.g., compositions comprising one or more bacterial species as described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Aced. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion or a powder. Various delivery systems are known and can be used to administer a composition of the disclosure, e.g. encapsulation in liposomes, microparticles, microcapsules, and the like, and then delivered to a patient by means of such as a nebulizer.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

The term “freeze-dried (lyophilized) as used herein refers to a preparation of bacterial cells that have been initially frozen and the water content removed by vacuum.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “preventing” means to stop or hinder a disease, disorder, or symptom of a disease or condition through some action.

The term “probiotic” is recognized in the state of the art as a microorganism which, when administered in adequate amounts, confers a health benefit to the host. A probiotic microorganism must fulfil several requirements related to lack of toxicity, viability, adhesion and beneficial effects. These probiotic features are strain-dependent, even among bacteria of the same species. Therefore, it is important to find those strains that have a better performance in all probiotic requirements.

The term “reducing” means to diminish in extent, amount, or degree.

The term “therapeutic agent” as used herein refers to a therapeutic substance selected from a group consisting of, but not limited to, analgesics, anesthetics, anti-inflammatory agents, antiasthma agents, antibiotics (including penicillins), anticoagulants, antihistamines, antitussives, antihypertensive agents, antimuscarinic agents, antimycobacterial agents, antioxidant agents, antipyretics, immunosuppressants, immunostimulants, antiviral agents, bacteriostatic agents, bronchodilators, buffering agents, contrast media, corticosteroids, cough suppressants (expectorants and mucolytics), diagnostic agents, diagnostic imaging agents, free radical scavenging agents, growth factors, haemostatics, immunological agents, lipid regulating agents, muscle relaxants, proteins, peptides and polypeptides, prostaglandins, radio-pharmaceuticals, time release binders, anti-allergic agents, stimulants and anoretics, steroids, sympathomimetics, vasodilators, and xanthines.

The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition.

### DISCUSSION

Enabling minimally invasive and precise control of liquid release in dental implants is crucial for therapeutic functions such as delivering antibiotics to prevent biofilm formation, infusing stem cells to promote osseointegration, and administering other biomedicines. However, achieving controllable liquid cargo release in dental implants remains challenging due to the lack of wireless and miniaturized fluidic control mechanisms. Here wireless miniature pumps and valves that allow remote activation of liquid cargo delivery in dental implants, actuated and controlled by external magnetic fields (<65 mT), are reported. A magnet-screw mechanism in a fluidic channel to function as a piston pump, alongside a flexible magnetic valve designed to open and close the fluidic channel, is proposed. The mechanisms are showcased by storing and releasing of liquid up to 52 μL in a dental implant. The liquid cargos are delivered directly to the implant-bone interface, a region traditionally difficult to access. On-demand liquid delivery is further showed by a metal implant inside both dental phantoms and porcine jawbones. The mechanisms are promising for controllable liquid release after implant placement with minimal invasion, paving the way for implantable devices that enable long-term and targeted delivery of therapeutic agents in various bioengineering applications.

Described herein are devices, systems, and methods relating to wirelessly actuated dental implants. Implants according to the present disclosure comprise a reservoir for storing a medicament that is to be delivered to a subject. According to aspects of the present disclosure, an actuator capable of generating a magnetic field can be utilized with aspects of the present disclosure to deliver a medicament to a patient.

### I. DENTAL IMPLANTS

Described herein are dental implants. In embodiments, dental implants as described herein can comprise an internal fluidic chamber extending axially through the implant. In embodiments, the internal chamber can be for storing a liquid and delivering the contents of the liquid in the presence of an external magnetic field.

In embodiments, dental implants as described herein can comprise one or more inner microfluidic channels. In embodiments, the one or more inner microfluidic channels can ne in fluidic communication with, and extend radially from, the internal fluidic chamber to one or more outer microfluidic channels.

In embodiments, dental implants as described herein can comprise one or more outer microfluidic channels. In embodiments, the outer microfluidic channels comprise a helical groove transversing outer threads of the implant.

In embodiments, dental implants as described herein can comprise an actuator. The actuator can be configured within the internal fluidic chamber. In embodiments, the actuator can be wirelessly actuated to pump liquid through the implant through application of an external magnetic field (a rotating magnetic field, for example). In embodiments, the actuator can comprise a magnet-screw piston. In embodiments, the actuator can comprise a screw bonded to a magnet. In embodiments, the screw comprises stainless steel. In embodiments, the magnet is a NdFeB magnet. In an embodiment, epoxy can bond the screw to the magnet. In embodiments, actuators can further comprise a biocompatible coating. In embodiments, the coating comprises PDMS and parylene-C. In embodiments, the stopper structure is coated with parylene-C. Un embodiments, the internal fluidic chamber comprises a small inlet, wherein the internal fluidic chamber can be refilled with the liquid. In embodiments, the implant is titanium. In embodiments, the flexible magnetic valve can be bonded to the stopper structure using uncured PDMS.

In embodiments, dental implants as described herein can comprise a flexible magnetic valve. In embodiments, the flexible magnetic valve can be configured between the internal fluidic chamber and the one or more inner microfluidic channels. In embodiments, the flexible magnetic valve can be opened or closed through application of an external magnetic field (for example, an external magnetic field axial to the actuator).

In embodiments, dental implants as described herein can comprise a stopper structure. In embodiments, the stopper structure can be bonded to the flexible magnetic valve. In embodiments, the stopper structure can maintain an opening angle or a closed position of the flexible magnetic valve after the axial external magnetic field has been removed.

### II. SYSTEMS

Described herein are systems. Systems as described herein can comprise any dental implant as described herein.

Systems as described herein further comprise a magnetic actuation unit. Magnetic actuation units according to the present disclosure can further comprise a magnet, step motor, battery, and microcontroller. In embodiments, the magnet is mounted onto a step motor. In embodiments, the step motor is controlled by a step motor driver. In embodiments, the battery can be connected to the step motor driver and microcontroller.

Magnetic actuation units according to the present disclosure can further comprise a switch. The switch can be connected to the microcontroller, for example, and allows a user to modify the speed of the step motor driver

Magnetic actuation units can generate a magnetic field. A magnetic field generated by the magnetic field actuation unit can be about 4 mT to 65 mT in strength. A magnetic field generated by the magnetic field actuation unit can be applied in a direction axial to the dental implant in order to open or close a flexible magnetic valve. In embodiments, the axially-applied magnetic field generated by the magnetic field actuation unit causes the flexible magnetic valve to open and remain in a locked open state by surmounting friction and mechanical stress posed by a stopper, allowing ejection of a liquid from within the internal fluidic chamber. In other embodiments, the axially-applied magnetic field generated by the magnetic field actuation unit causes the flexible magnetic valve to close and remain in a closed state by overcoming resistance from the stopper. A rotating magnetic field generated by the magnetic field actuation unit can also be applied in a direction radial to the dental implant in order to wirelessly actuate an actuator within the internal fluidic chamber of the implant. In embodiments, the rotating magnetic field generated by the magnetic field actuation unit causes a magnet-screw piston to rotate within the internal fluidic chamber of the implant and translate axially within the internal fluidic chamber, controlling ejection of liquid from the internal fluidic chamber along with the flexible magnetic valve. In embodiments, the magnetic actuation unit generates the external magnetic field, controlling delivery of the liquid through the implant.

### III. KITS AND PACKAGING

Described herein are kits comprising dental implants and systems comprising such, in addition to containers for storage and/or use, and instructions for use. The kit can further comprise one or more medicaments for loading into the dental implant. The kit can be a package which houses a container which contains components of the disclosure or formulations of the disclosure and also houses instructions for administering the compounds or formulations to a subject. The disclosure further relates to a commercial package comprising compounds of the disclosure or formulations of the disclosure together with instructions for simultaneous, separate or sequential use. In particular a label may include amount, frequency, and method of administration. In embodiments, for example, a system comprises a dental implant, an actuation unit, and instructions for use (i.e., instructions for implanting the dental implant, loading the dental implant with one or more medicaments, and the like).

The disclosure also provides a pharmaceutical pack or kit comprising one or more containers filled with one or more of the ingredients of a composition of the disclosure to provide a therapeutic effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The disclosure also relates to articles of manufacture and kits containing materials useful for treating a disease disclosed herein. An article of manufacture may comprise a container with a label. Examples of suitable containers include bottles, vials, and test tubes, or a delivery device such as a nebulizer, which may be formed from a variety of materials including glass and plastic. A container holds compounds of the disclosure or formulations of the disclosure which are effective for treating a disease disclosed herein. The label on the container indicates that the compounds of the disclosure or formulations of the disclosure are used for treating a disease disclosed herein and may also indicate directions for use. In aspects of the disclosure, a medicament or formulation in a container may comprise any of the medicaments or formulations disclosed herein.

The disclosure also contemplates kits comprising one or more of compounds of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein). A medicament or formulation in a kit of the disclosure may comprise any of the formulations or compositions disclosed herein.

The compositions, for example, an antibiotic composition, can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

A composition as described in this disclosure for use in treating a bacterial infection in subjects may be delivered in a pharmaceutical package or kit to doctors and patients. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a composition or components of compositions described herein. In some embodiments, the pharmaceutical package or kit further comprises unit dose forms of one or more of an additional therapeutic, for example, another medicament used for treatment of a disorder in a patient.

In one embodiment, the kit or pharmaceutical package comprises a composition as described herein in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### IV. PHARMACEUTICAL COMPOSITIONS

Compositions comprising therapeutics are contemplated according to the present disclosure. The therapeutics can be, for example, small molecule therapeutics (i.e., those less than 2500 daltons, such as antibiotics), nucleotide therapeutics (i.e., compositions comprising one or more nucleic acids, for example, DNA and RNA), protein therapeutics, and stem cell therapeutics. The therapeutics may also comprise coating agents, bioagents, and other minimally invasive agents. The compositions may further comprise a diluent, solubilizer, emulsifier, preservative, and/or adjuvant to be used with the methods disclosed herein.

In embodiments, hydroxyapatite and other coating materials in liquid forms can be utilized with dental implants according to the present disclosure. In embodiments, antibiotics, such as Doxycycline, Amoxicillin and others in liquid forms can be utilized with dental implants according to the present disclosure. In embodiments, bioagents can be utilized, for example, stem cells for osseointegration (pluripotent cells such as iPSCs that can be differentiated in the subject, or differentiated prior to use in a dental implant according to the present disclosure, or multipotent cells such as mesenchymal stem cells).

### V. METHODS OF USE AND TREATMENT

As described herein, the present disclosure provides a method of treating a subject with a disorder characterized by a bacterial infection, particularly a recurring or treatment-resistant infection, (i.e., a subject “in need thereof”), comprising administering to the subject a therapeutically effective amount of compositions (i.e., those comprising, consisting essentially of, or consisting of therapeutics described herein) according to the present disclosure. In embodiments, a subject may have or be suspected of having peri implantitis or peri implant mucositis.

In embodiments, methods as described herein can comprise implanting any dental implant described herein into the jawbone of a patient. The implant can comprise a medicament so that opening a flexible magnetic valve within the implant is by application of an axial external magnetic field. In embodiments, the application of the magnetic field activates an actuator within the implant. In embodiments, the actuator then can pump the medicament through the flexible magnetic valve and out of the implant by application of a rotating external magnetic field, thereby delivering the medicament from the implant directly to the implant-bone interface.

Methods of the present disclosure can further comprise refilling the implant with the medicament. In embodiments, the implant is refilled by injecting the medicament into a small inlet of the implant. In embodiments, the medicament can comprise antibiotics, stem cells, drugs, or a combination of any thereof.

In embodiments, the actuator can be further configured to translate along the axis of the internal fluidic chamber when the rotating magnetic field is applied. In embodiments, the actuator can be further configured to translate along the axis of the internal fluidic chamber when the rotating magnetic field is applied by the magnetic actuation unit.

The compositions described herein are useful in, inter alia, methods for treating a bacterial infection in a subject. In some embodiments, the subject has or is suspected to have a bacterial infection. In some embodiments, the subject is diagnosed with a bacterial infection. In some embodiments, the subject is a human that is suspected of having a bacterial infection.

Treating or treatment of any disease or disorder refers to ameliorating a disease or disorder that exists in a subject or a symptom thereof, in particular, ameliorating symptoms of bacterial infection or bacterial toxin levels. The term ameliorating refers to any therapeutically beneficial result in the treatment of a disease state, e.g., a reduction in bacterial toxin levels or improved survival.

Thus, treating or treatment includes ameliorating at least one parameter or symptom. Treating or treatment includes modulating the disease or disorder, either physically (e.g., stabilization of a discernible symptom) or physiologically (e.g., stabilization of a physical parameter) or both. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a bacterial infection in a subject by administering a composition as described in this disclosure is considered to be a treatment or therapeutic, for example, if there is a 10% improvement according to the measured or observed parameter in a subject as compared to a control. Thus, the reduction can be a 10%, 20%, 30%, 40%, 50%, 60%, 70%, 80%, 90%, 100%, or more (or any percent improvement in between 10% and 100%) as compared to native or control levels. It is understood that treatment does not necessarily refer to a cure or complete ablation of the disease, condition, or symptoms of the disease or condition.

Described herein are therapeutically effective amounts as defined previously. A therapeutically effective amount is not, however, a dosage so large as to cause adverse side effects. A suitable dose capable of ameliorating a bacterial infection in a subject, can depend on a variety of factors including the particular construct used and whether it is used concomitantly with other therapeutic agents. Generally, a therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication.

A pharmaceutical composition can include a therapeutically effective amount of a therapeutic described herein. Such effective amounts can be readily determined by one of ordinary skill in the art as described above, embodiments are given throughout the present disclosure.

Suitable human doses of any of the therapeutics described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such micro-organisms can be determined by known pharmaceutical procedures in cell cultures or experimental animals (e.g., animal models of any of the diseases described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A composition comprising therapeutics that exhibits a high therapeutic index is preferred. While constructs that exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of a therapeutic lies generally within a range of circulating concentrations of the therapeutics that can include the ED₅₀ with little or no toxicity. The dosage may vary within this range depending upon the dosage form employed and the route of administration utilized. For therapeutics herein, the therapeutically effective dose can be estimated initially from cell culture assays. A dose can be formulated in animal models to achieve a circulating plasma concentration range that includes the EC₅₀ (i.e., the concentration of the construct—e.g., antibody—which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

In some embodiments, a composition or component of a composition described herein (i.e., one or more bacterial strains comprising micro-organisms) can be administered to a subject as a monotherapy. Alternatively, the composition or component of a composition described herein can be administered in conjunction with other therapies for the bacterial infection (combination therapy). For example, the composition can be administered to a subject at the same time, prior to, or after, a second therapy. In some embodiments, the composition or component of a composition described herein, and the one or more additional active agents are administered at the same time. Optionally, the composition or component of a composition described herein is administered first in time and the one or more additional active agents are administered second in time. In some embodiments, the one or more additional active agents are administered first in time and the micro-organism composition or component of a composition described herein is administered second in time. Optionally, the composition or component of a composition described herein, and the one or more additional agents are administered simultaneously in the same or different routes.

A composition as described herein can replace or augment a previously or currently administered therapy, such as previously prescribed antibiotic.

Monitoring a subject (e.g., a human patient) for an improvement of symptoms of bacterial infection, as defined herein, means evaluating the subject for a change in a given parameter or symptom exhibited by the subject by a clinician in a social setting or self-reporting by the patient. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality.

In certain embodiments, the effective amount of a pharmaceutical composition comprising one or therapeutics of the present disclosure to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a micro-organism is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the active components in the formulation used. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### VI. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: Wirelessly Actuated Microfluidic Pump and Valve for Controlled Liquid Delivery in Dental Implants

### A. Introduction

On-demand and precise liquid cargo delivery in implants[1,2] holds the promise of administering therapeutic agents, such as antibiotics to prevent biofilm formation or stem cells for tissue regeneration, in a controlled manner over prolonged durations.[3,4] It also allows minimizing the dosage[5] and offering tailored, controllable treatments with reduced adverse effects.[6] Such advancements mark a necessary stride toward personalized and minimally invasive therapeutic interventions. In addition, the capability to release drugs in response to biological or external stimuli[6,7] is also crucial for responsive, on-demand, and wirelessly controlled therapy.[8] Dental implants as one example, widely utilized as substitutes for tooth roots, are surgically embedded within the alveolar bone. Unfortunately, infections and inflammation due to bacterial colonization affecting the gum tissue, bone, and surrounding connective tissues[9,10] are prevalent issues that can lead to implant loosening after oral surgeries. These complications encompass a range of issues, including screw or abutment loosening, deterioration of access hole restorations, minor chipping and fractures, soft tissue complications, bone loss, and peri-implant infections.[11]

Developing advanced technologies and strategies to hinder bacterial colonization on implant surfaces,[12,13] holds immense promise in significantly reducing the prevalence and impact of peri-implant mucositis over the long term.[3] Coating dental implants presents a promising avenue for inhibiting bacterial growth, yet the long-term stability of these coatings remains a concern due to unavoidable degradation and coating damage during implant installation.[14] Various methods exist for gradually releasing antibiotics, such as coating the implant with specific materials to impede biofilm formation or embedding antibiotics within porous structures in the implant itself.[15] Previous research has explored passive drug release via coating[16] or drug dispensation within the implant chamber[17] based on diffusion. However, traditional chemical coating approaches, involving antibiotics and antibacterial peptides applied to modified titanium surfaces, exhibit a rapid decline in antibacterial potency over time. Consequently, achieving sustained antibacterial efficacy with conventional dental implants proves challenging. In addition, employing stem cells to foster osseointegration between dental implants and surrounding bone tissue at the early stage holds considerable promise.[18] However, achieving controlled delivery of stem cells also remains a daunting task.

Active and on-demand drug or stem cell release mechanisms have yet to be demonstrated in dental implants, primarily due to the absence of wireless and miniaturized pumping mechanisms. While limited controllable release of antibiotics for peri-implant mucositis prevention has been explored in previous studies, passive antibiotic release via drug-filled or coated implants has been the primary focus.[13,19] For instance, mesoporous silicon dioxide matrices incorporated into titanium implant walls enable passive drug release[20] by diffusion. Additionally, swelling-based triggerable drug release mechanisms utilizing environmental moisture[21,22] have been investigated, but controlling the release rate proves challenging. Lastly, existing polymer pumps and valves[23-26] are either too big or cannot be wirelessly controlled preventing their applications in targeted delivery of antibiotics or stem cells and other bioagents in liquid form in dental implants. For example, radio frequency (RF) magnetic field[26] has been employed to induce an electrical voltage to actuate a cantilever beam made of ionic polymer-metal composite for drug release but the actuation distance is limited to only several millimeters. In summary, achieving both on-demand and controllable liquid cargo release in bone implants remains challenging due to the lack of wireless and miniaturized fluidic control mechanisms.

The present disclosure introduces a fundamental mechanism for wirelessly controlled liquid cargo delivery within dental implants, achieved through the seamless integration of wireless microfluidic channels, pumps, and valves. First, the present disclosure presents a magnetic screw-based piston and a flexible magnetic valve, allowing the precise release of liquids on demand, all controlled by external magnetic fields. Second, the functionality of the valve and pump within a dental implant is showcased and a handheld magnetic actuation system designed to effortlessly integrate into routine tooth cleaning procedures is introduced. Lastly, to validate the technology, experiments in both phantoms and animal bones were conducted, confirming the efficacy of the wirelessly controlled pumping and valving mechanisms. This wireless, controllable drug release capability holds immense promise for providing efficient and long-lasting treatment for inflammation, eliminating the necessity for invasive surgeries. Beyond existing pumping mechanisms, the present disclosure has the unique capability of fully wirelessly controlled pumping of liquid cargos seamlessly integrated with existing dental implants. This method allows delivery of liquid cargos that are challenging using injection methods and could allow renewing coating agents, and delivery of various bioagents on demand, paving the way for minimally invasive therapeutic functions in dental implants.

### B. Overview of the Wirelessly Controlled Liquid-Delivery Dental Implant

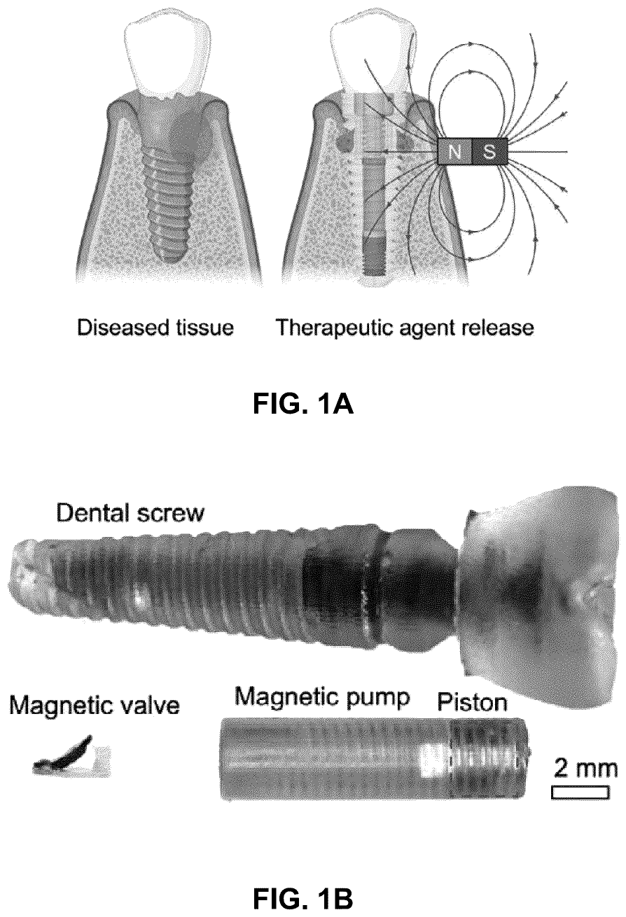

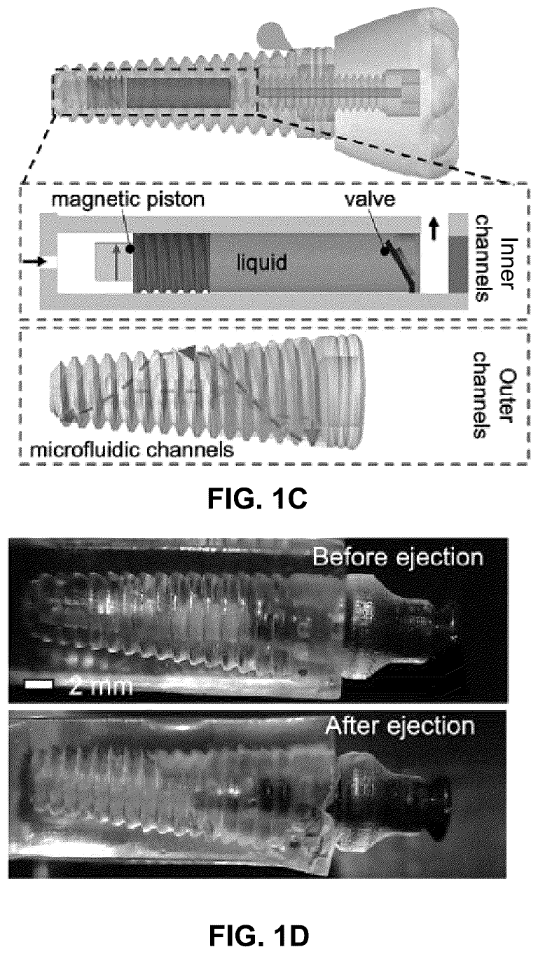

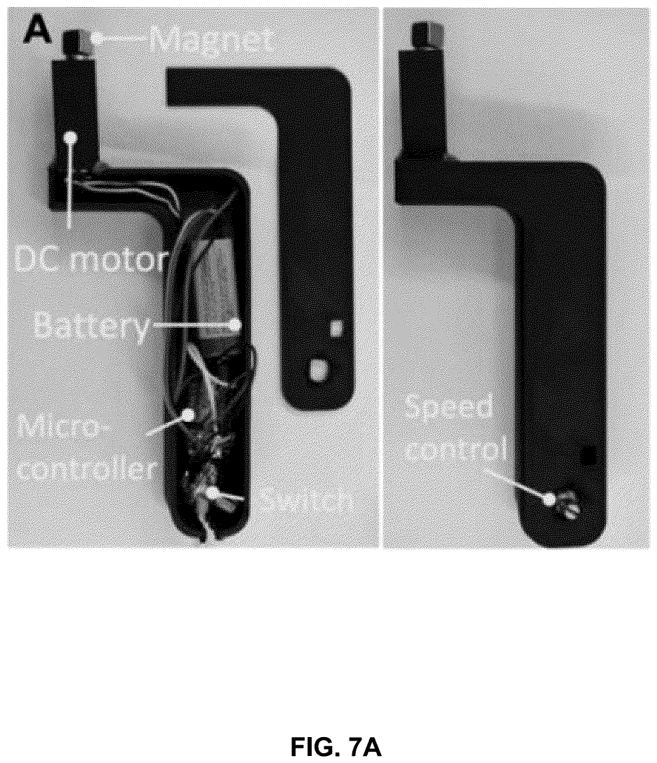

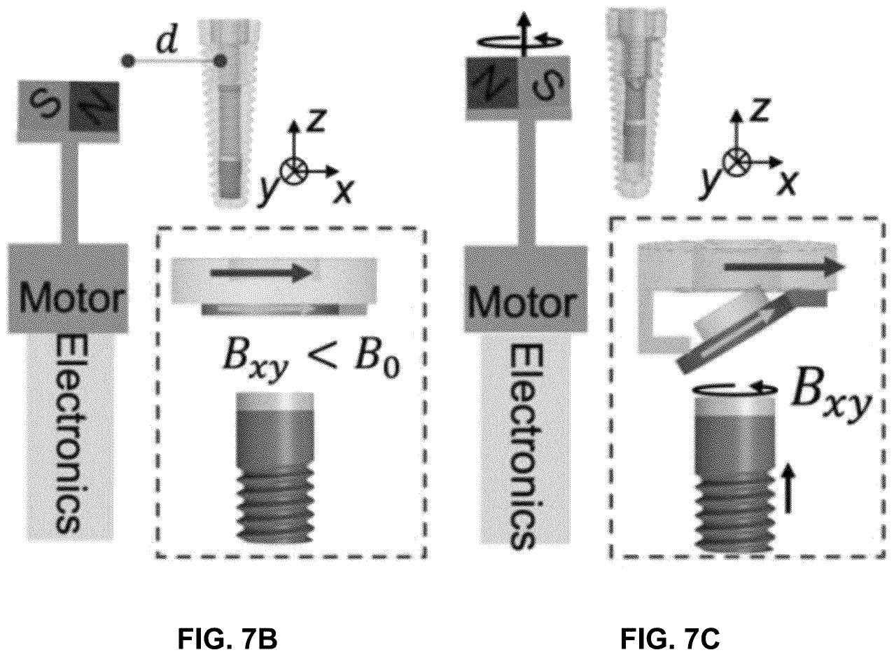

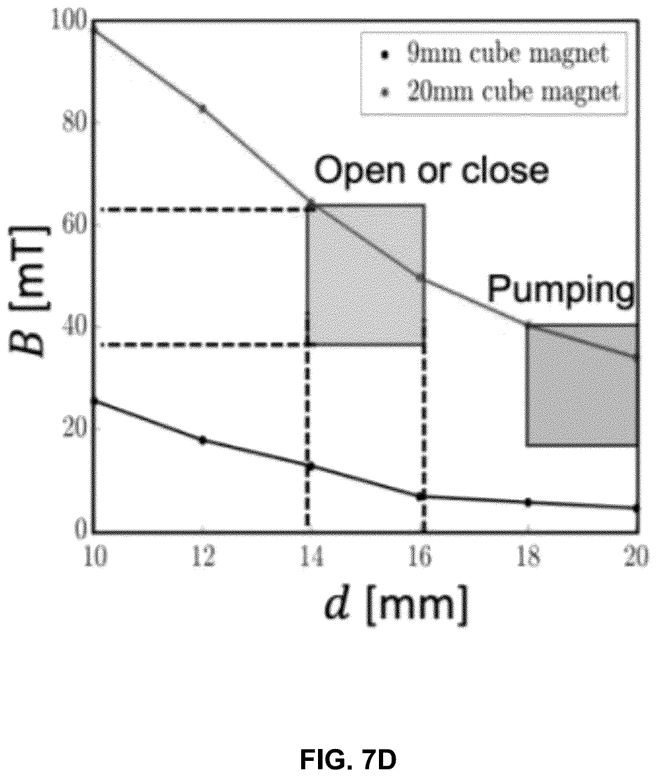

As shown in FIG. 1A, when filled with liquid cargos such as antibiotics, the piston can be remotely actuated by external magnetic fields to eject the liquid drug out of the dental screw. A major advantage of the proposed dental implant is its ability to allow on-demand release of liquid cargos with minimal invasion, as the magnetic field can easily penetrate biological tissues and non-magnetic metal materials such as titanium.[27] Various types of liquids, such as antibiotics or solutions with stem cells, can be filled into the reservoir for long-term use. The dental implant system, illustrated in FIG. 1B, comprises a dental screw with inner fluidic channels, a magnet-screw piston, and a flexible magnetic valve. The flexible magnetic valve can be remotely controlled to open, allowing the liquid drug to be released from the chamber outlet, and to close, preventing leaks. Additionally, both the pumping and valving operation in the dental implant are controlled by external magnetic fields using a handheld magnetic actuation unit (FIGS. 7A-7D).

FIG. 1C illustrates the fundamental working principle of the device. The magnet-screw piston comprises a NdFeB magnet (1 mm by 1 mm by 1 mm) bonded to a miniature screw (diameter: ≈1.9 mm, length: 2 mm) to form a magnetic propeller. The bonded magnet has a radial magnetic moment that can be rotated by applying a rotating magnetic field, allowing the magnetic piston to translate along the axis of the internal chamber. The internal channel of the dental implant is designed to store liquid cargos. The grooved pattern, helical surface of the dental screw, and surrounding material form the fluidic channels to distribute the liquid cargo. FIG. 1D shows an example of releasing liquid with fluorescent dyes inside a dental phantom. The dental implant enables the release of liquids to wet the surface of the implant, providing a relatively large coverage area.

### Example 2: Fabrication of the Wirelessly Actuated Fluidic Modules

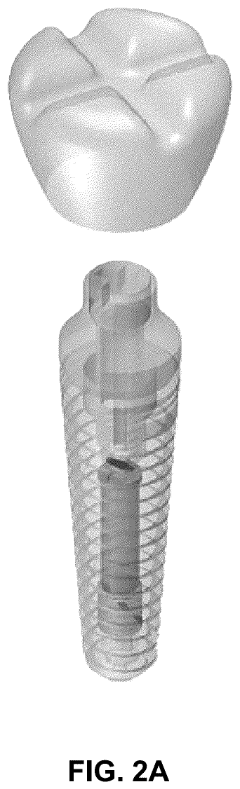

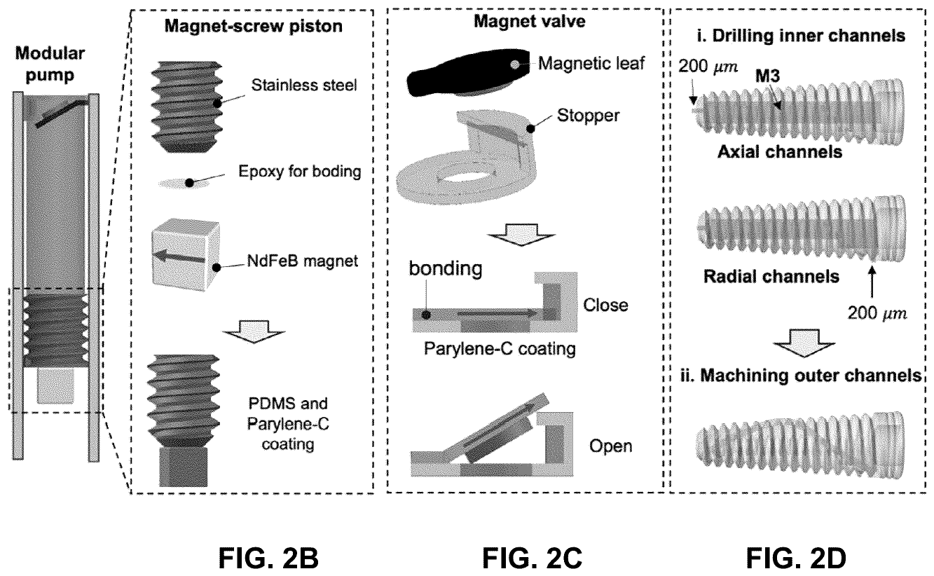

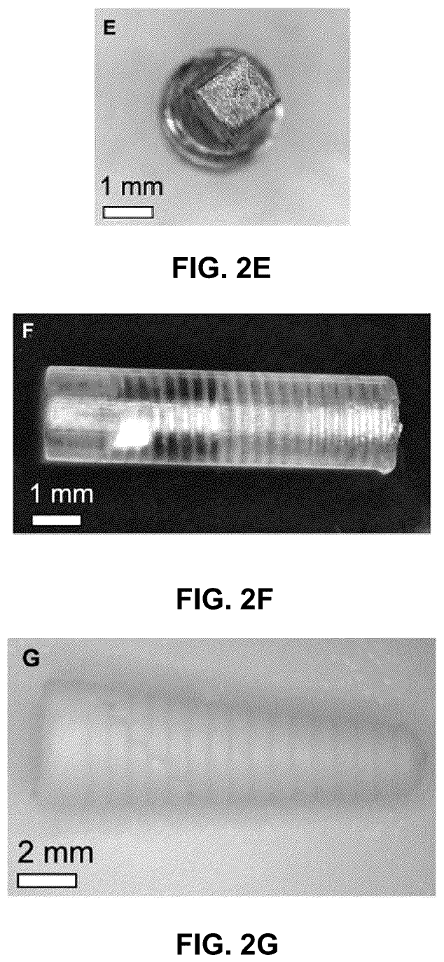

The present disclosure shows the design and fabrication process of the dental implant with integrated fluidic modules in FIGS. 2A-2G. FIG. 2A provides an overview of the dental implant, showcasing all integrated components, including the modular pump, dental screw with fluidic channels, and the abutment. First, the modular pump is fabricated following the process shown in FIG. 2B, with more details described in “Fabrication of the Modular Piston Pump” of the “Experimental Section.” Briefly, the modular pump comprises a cylindrical tube with an internally tapped surface and a magnet-screw piston. The tube is 3D printed using UV-curable resin (Clear resin V4, Formlabs, Inc.) in a stereolithography (SLA) 3D printer (Form 3+, Formlabs, Inc.). The cylindrical tube is subsequently tapped using an M2 by 0.4 mm-tapping tool. The piston is assembled by bonding an M2 by 2 mm set screw with an NdFeB magnet (1 mm by 1 mm by 1 mm, Supermagnet, N45), which has a magnetic moment perpendicular to the screw body axis. After assembly, the magnet-screw piston is coated with polydimethylsiloxane (PDMS) and parylene-C to ensure biocompatibility (or other biocompatible materials such as polytetrafluoroethylene, polyethersulfone, titanium, and ceramics). Oil is utilized to seal the gap between the piston and the cylindrical tube. The spinning motion of the magnet-screw induces the translation of the piston along the main fluidic channel, facilitating the ejection of liquid from inside to outside.

Moreover, the fabrication process of the flexible magnetic valve is described in FIG. 2C and “Fabrication of the Lockable Valve” of the “Experimental Section,” using a combination of laser machining and assembly techniques. Briefly, FIG. 2C shows that the magnetic leaf is prepared through laser cutting, while the stopper and outer fixture are prepared by molding. Subsequently, the leaf and fixture are securely bonded using uncured polymer, with a PDMS plug facilitating effective sealing. In an embodiment, the magnetic leaf comprises a magnetic composite made of NdFeB Microparticles and Ecoflex 00-30 elastomer. The lockable valve assembly involves integrating a foldable magnetic leaf comprising Ecoflex 00-30 (or Ecoflex 00-50, Ecoflex 00-10, for example) and NdFeB magnetic particles, a base layer (PDMS), and the locking mechanism (PDMS). The flexible magnetic leaf is magnetized in a specific direction (denoted as a red arrow in FIG. 2C) in an impulse magnetizer (IM-10-30, ASC Scientific) with a 1.8 T magnetic field impulse. The assembly process involves manual bonding of the magnetic leaf and locking mechanism using uncured PDMS, ensuring precise integration of components.

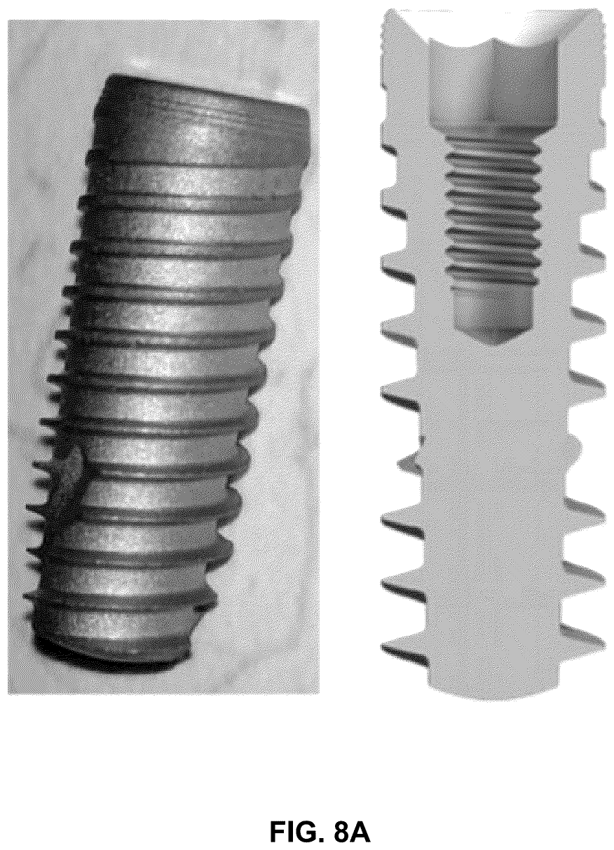

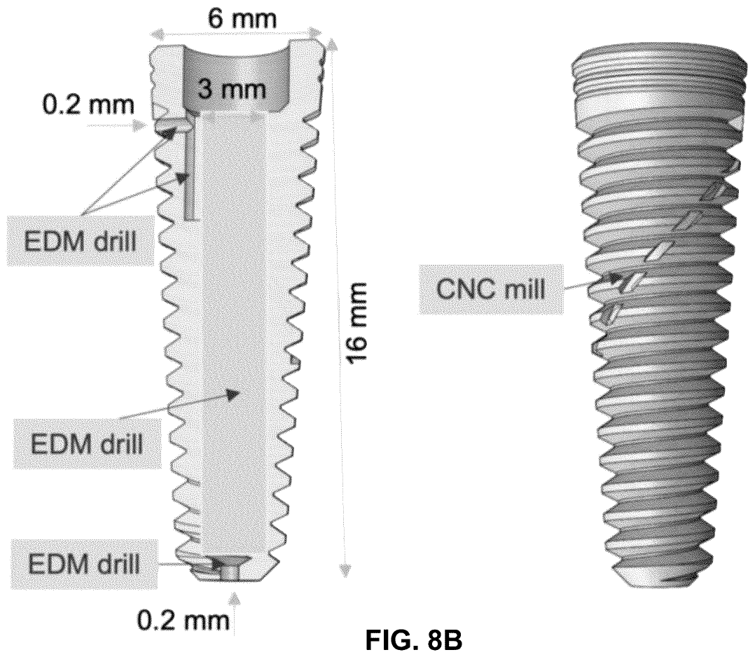

Lastly, FIG. 2D illustrates the fabrication process of the dental screw. The fluidic channels and grooved patterns of the dental screw play crucial roles in facilitating liquid dispersion. The fluidic channel comprises an inner chamber for liquid storage and a microfluidic channel connecting it to the dental implant's outer surface via the outlet. This outlet location enables the liquid to follow the helical structure and groove pattern, effectively wetting the surface when a gap is present. Additionally, this outlet position is closer to the top part of the dental screw, where inflammation is more commonly observed. The grooved pattern mitigates circular pressure exerted by surrounding materials. To fabricate the screw with fluidic channels, a dental screw is either 3D printed or commercially obtained, followed by tapping and drilling processes to create the desired channels. If metal machining is employed, modifications to commercial dental implants are made, with drilling and tapping executed using electrical discharge machining (EDM) and computer numerical control machining (FIGS. 8A-8B). FIGS. 2E-2G sequentially show the magnet-screw piston, the modular pump with the magnetic piston inserted into the threaded tube, and the dental screw featuring a grooved helix on its outer surface.

### Example 3: Characterization of the Magnetically Actuated Piston Pump

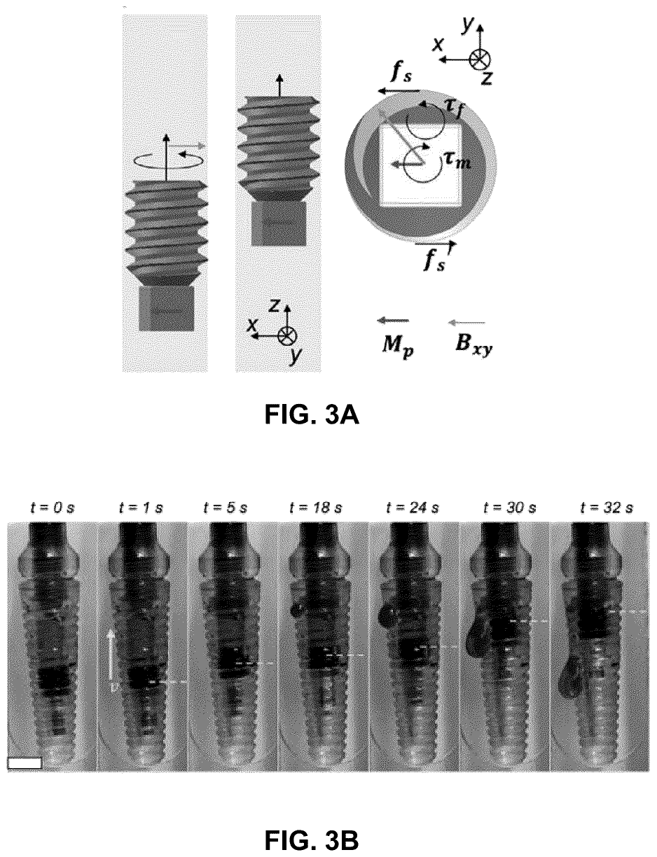

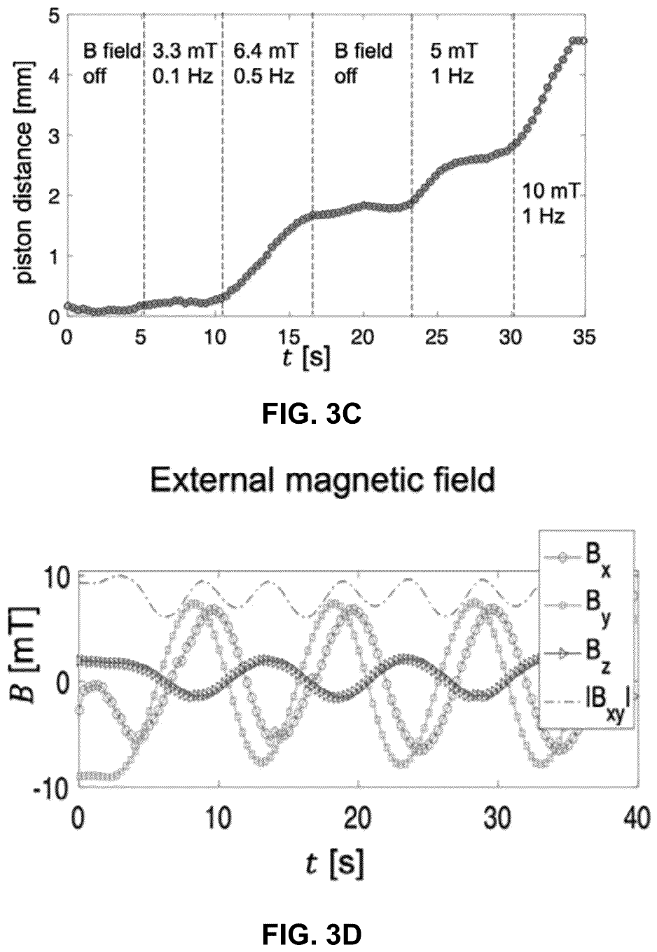

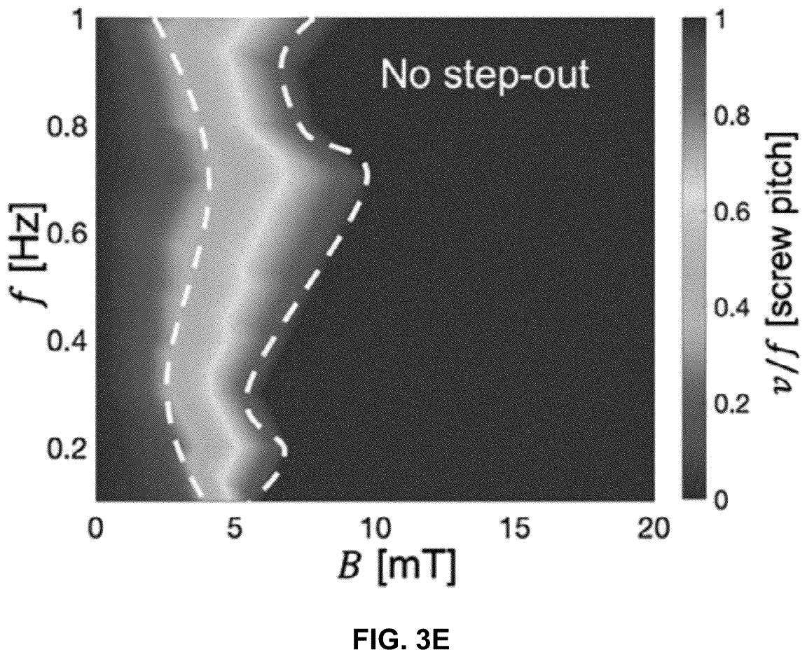

FIGS. 3A-3E characterize the liquid ejection performance controlled by external magnetic fields. As shown in FIG. 3A, the speed of liquid ejection is finely regulated by adjusting the frequency of the applied magnetic field. In FIG. 3B, successful liquid ejection is demonstrated when the magnetic piston is rotated, using Deionized (DI) water mixed with blue food dye to emulate a liquid drug. FIG. 3C illustrates the distance traveled by the piston over time under a rotating magnetic field (B=10 mT). When a relatively small magnetic field is applied, the friction prevents the piston from rotating at the same frequency as the external magnetic field, resulting in step-out motion. However, by increasing the magnetic field strength to ≈10 mT, as depicted in FIG. 3D, continuous liquid ejection is achieved.

In addition, the piston motion was assessed under various frequencies and magnitudes of a rotating magnetic field. FIG. 3E shows the piston speed divided by the magnetic field frequency given by v/f. Notably, when the magnetic field is relatively small, the step-out frequency of the piston is relatively small. Factors such as fluid pressure and friction can induce piston step-out,[28] causing it to deviate from following the external magnetic field, particularly under conditions of weak magnetic fields or high frequencies. Therefore, a larger magnetic field at a lower frequency is preferable to facilitate controlled liquid ejection. Increasing the magnetic field strength allows the piston to overcome friction and accurately track the external magnetic field. It is essential to maintain the magnetic field strength above a certain threshold to prevent friction-induced step-out of the rotational motion. This ensures precise control of drug release volume, preventing unintended deviations in the delivery process.

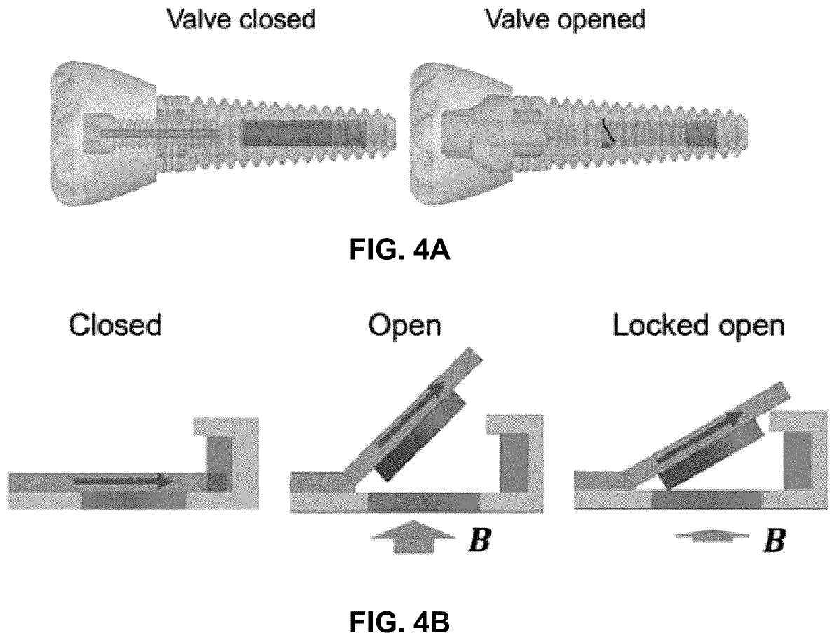

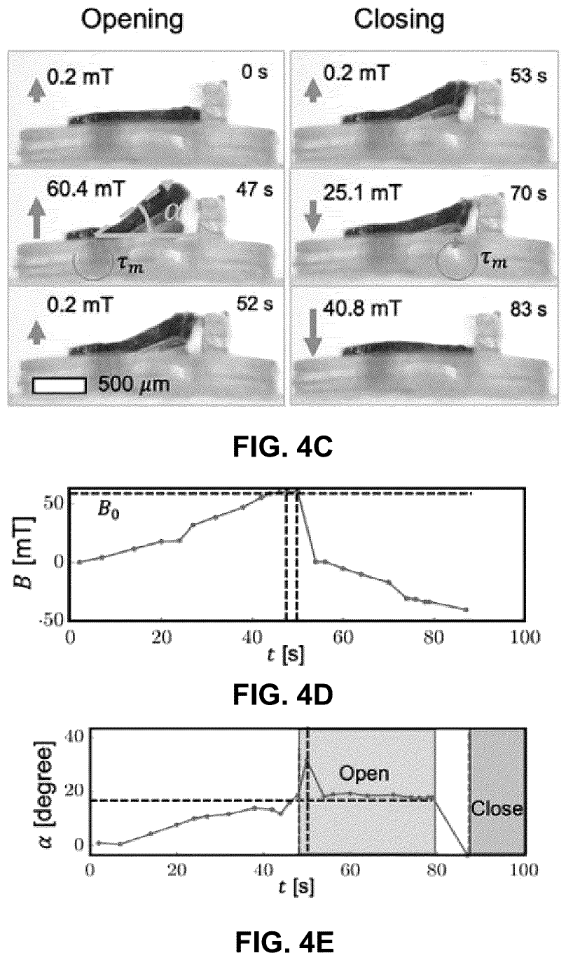

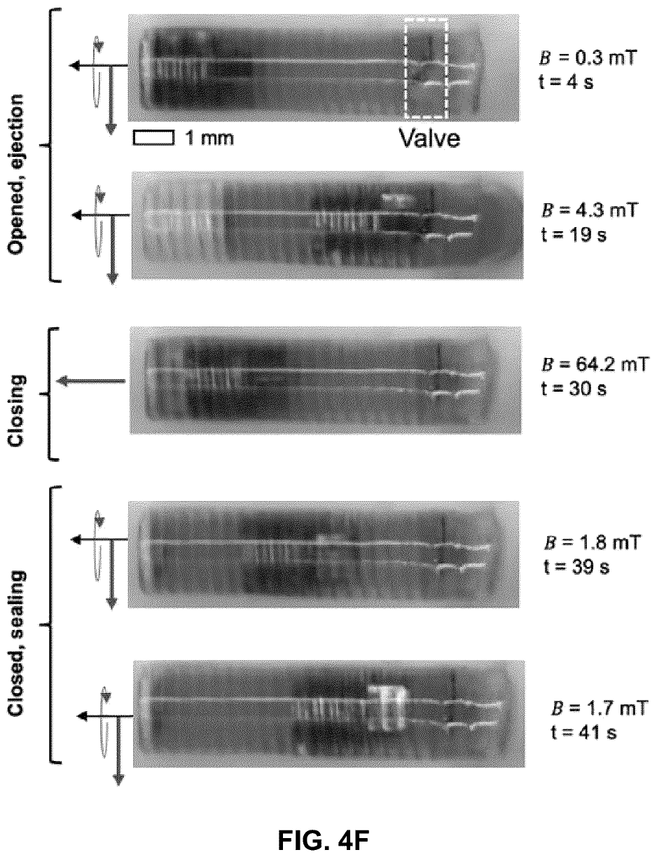

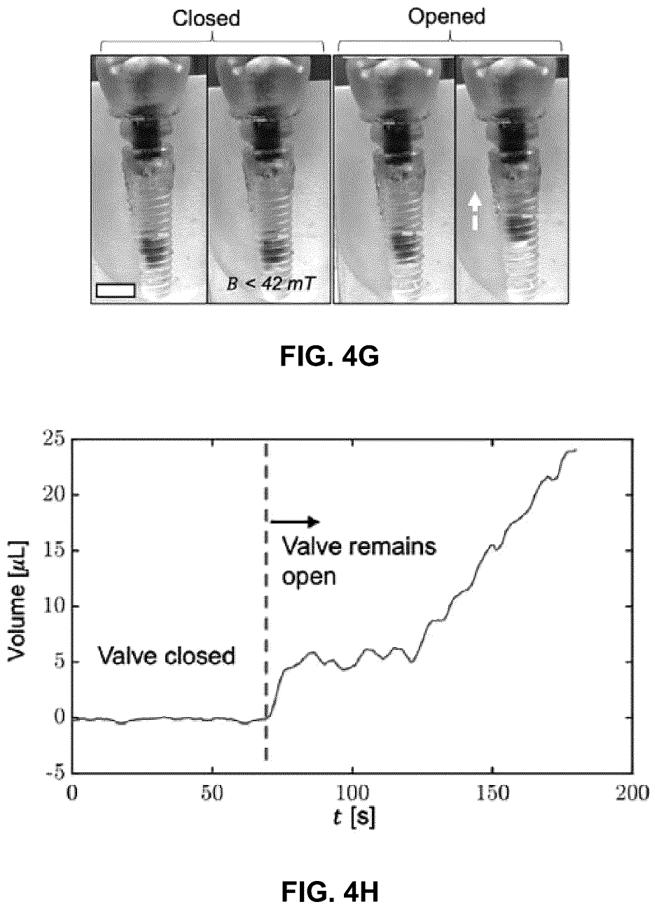

To characterize the valve control mechanism, FIGS. 4A, 4B show that the valve is designed to open upon the application of a substantial magnetic field in the axial direction of the dental screw. Constructed from magnetic composite materials as detailed in prior studies,[29,30] the valve exhibits the function of remotely controlled opening and closing. As shown in FIG. 4B, when a magnetic field of a relatively large magnitude is applied axially, the valve opens by surmounting friction and mechanical stress posed by the stopper. Once past the stopper, the valve remains in a locked open state, maintaining its position even under relatively weak magnetic fields. Conversely, applying a larger magnetic field in the reverse direction prompts the valve to dose, overcoming resistance from the stopper. The presence of hysteresis enables the valve to open during piston rotation, often requiring a smaller magnetic field.

FIG. 4C illustrates the locking mechanism designed to maintain the valve in an open position. A magnetic field is applied in the +y direction, as depicted in FIG. 4D. The valve includes a magnetic body possessing a magnetic moment. Initially, when a small magnetic field (<60.4 mT) is applied, the valve remains closed. However, once the magnetic field exceeds a threshold value, denoted as B0, the valve opens, achieving a relatively large angle α due to magnetic torque,[31] as shown in FIG. 4E. The critical magnetic field B0 could be adjusted as demanded by varying the magnetization and other material properties of the valve. Moreover, the valve incorporates a locking mechanism to ensure sustained opening, even with reduced magnetic field strength. The valve will only dose upon reversing the magnetic field and incrementally increasing its strength, as the magnetic torque becomes sufficiently strong to overcome the friction.

To further enable on-demand drug releasing and sealing, the magnetic valve and pump are coordinately controlled as shown in FIGS. 4F-4H. First, FIG. 4F shows a modular pump with an integrated valve to demonstrate the sealing ability. When a rotating magnetic field of ≈4.3 mT is applied, the piston pump could eject liquid as desired when the valve is open. When a static magnetic field of 64.2 mT is applied, the valve is closed. A rotating magnetic field could move the piston but cannot eject the liquid out due to the sealing. Similarly, FIGS. 4G and 4H show that in another fully assembled 3D printed dental implant, the valve remains closed with a as nearly zero degree when a magnetic field of less than 60.4 mT is applied. When the magnetic field surpasses this value, the magnetic valve remains open. Then, the rotating magnetic field enables transportation of liquid outside of the internal chamber. To close the valve, a magnetic field of ≈64.5 mT is then applied to push the polymer plug into the hole for sealing. To further quantify the valve control on fluid pumping control, the liquid ejection volume was plotted as a function of time in FIG. 4G when the valve and piston pump are coordinated to control the ejection of liquid.

### Example 4: Characterization of Liquid Election Inside Phantoms by the Dental Implant

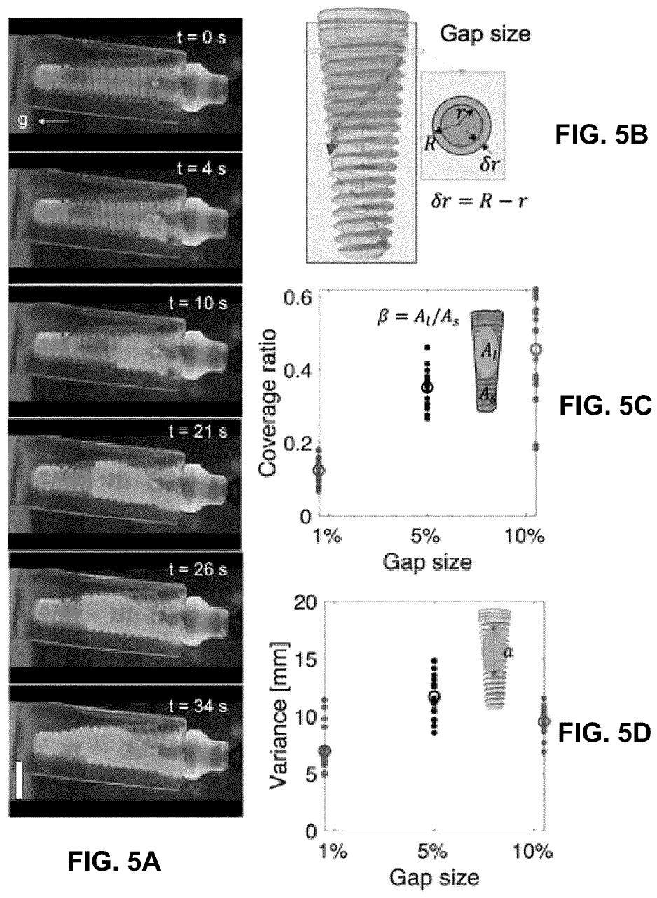

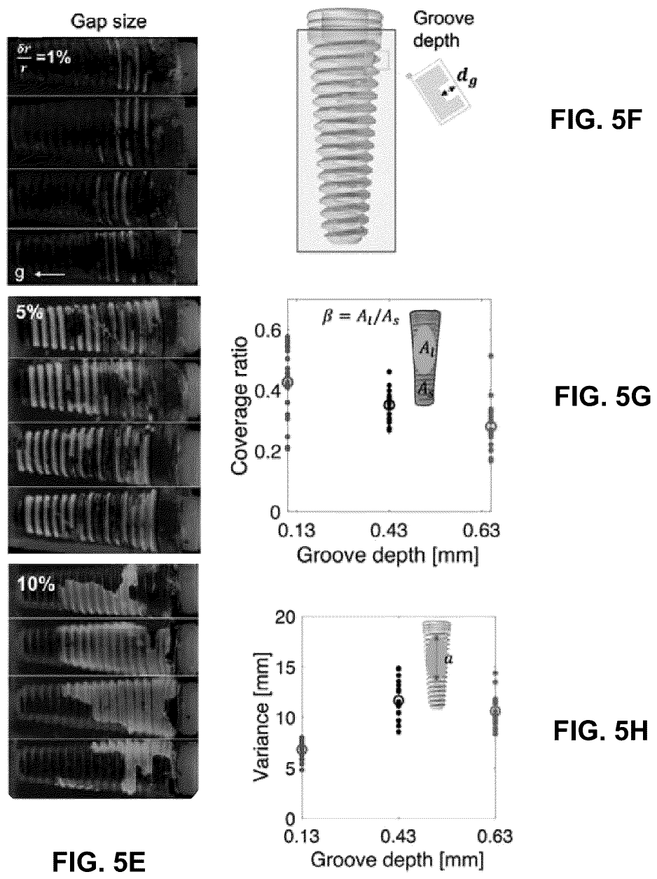

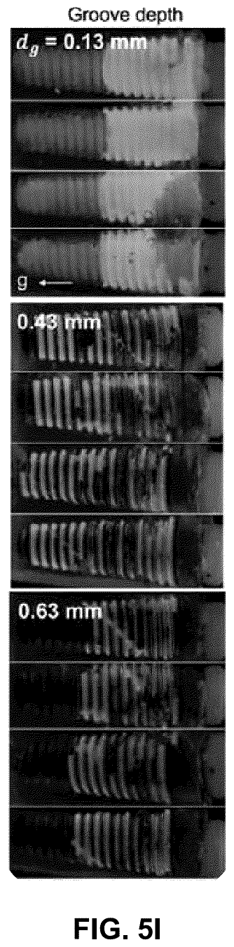

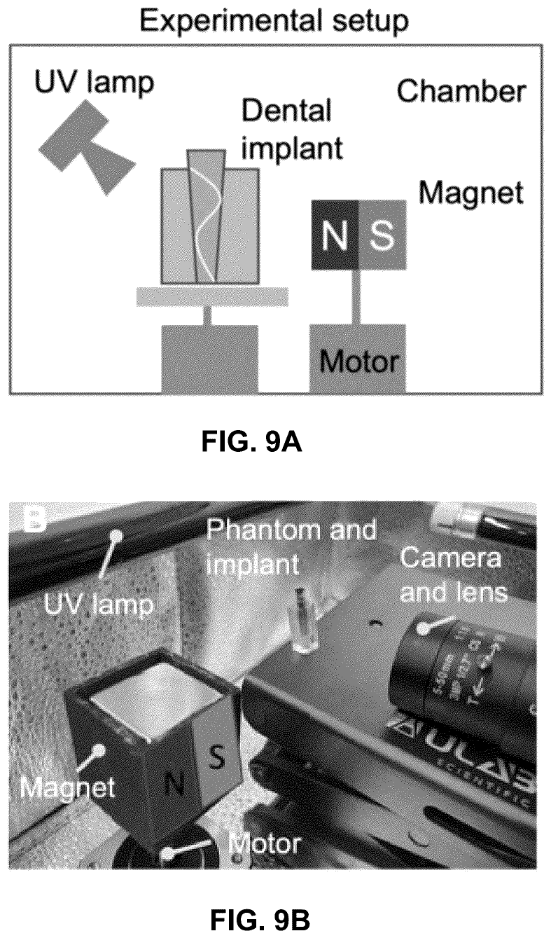

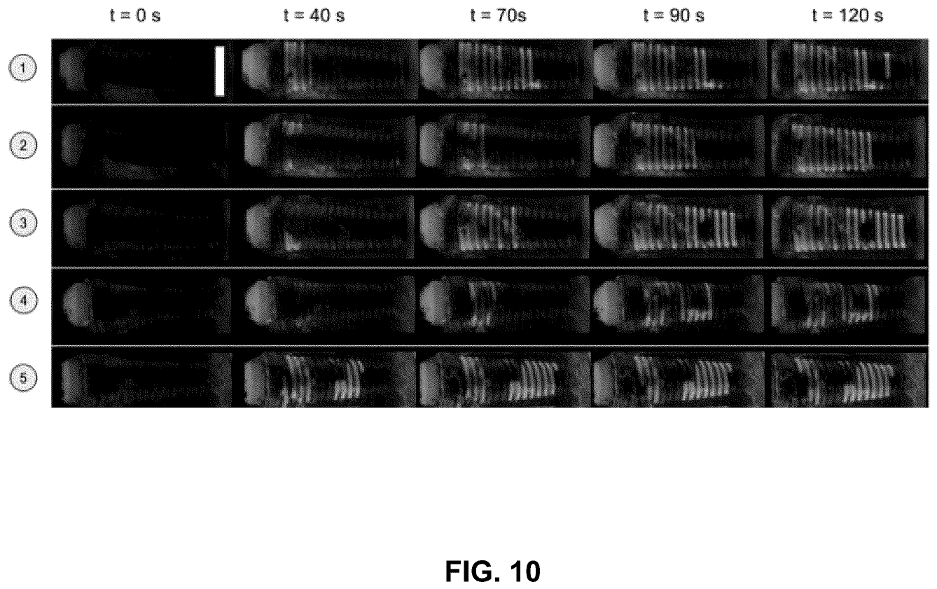

Peri-implant diseases, mainly peri-mucositis and periimplantitis, with incident rates ranging from 24% to 88% and 10% to 47%, respectively,[32,33] stand as a primary cause of dental implant restoration failure, especially among individuals with uncontrolled diabetes mellitus[34] and certain genetic disorders.[35] Preventing bacterial colonization on the implant surface is imperative for long-term prevention of peri-implant mucositis and peri-implantitis. In FIGS. 5A-5I, the liquid release capability of a dental implant placed within a phantom of varying gap sizes to emulate the stages of peri-implant diseases was investigated. FIG. 5A illustrates the placement of a dental implant within a transparent PDMS phantom, chosen for its transparency, revealing the inner cavity molded with dental screws of various diameters. To ensure visibility under UV light (wavelength: 365 nm), the liquids are infused with fluorescent dyes and the dental screw is prepared by transfer molding instead of direct 3D printing. During testing, these samples are housed within a black box, with external UV light control as shown in FIGS. 9A-9B. Initially, at t=0 s, no fluid is observed on the outer surface of the dental implant. Subsequently, a rotating magnetic field with parameters B=15 mT and f=2 Hz is applied. By t=34 s, the fluorescent liquids are fully expelled from the inner chamber as the magnetic piston ascends to the top position. To ensure the repeatability, for each dental screw design and a phantom design, five trials of liquid ejection are performed as showcased in FIG. 10.

To evaluate the wetting capabilities of the dental implant's surrounding surface, the impact of the gap size between the implant and the surrounding materials on liquid coverage was investigated. Illustrated in FIG. 58, the parameter δr denotes the gap size, representing the variance in radius between the phantom and the dental implant, thus simulating diseases at various stages. Tests were conducted with dental implants of different sizes within phantoms and subsequently segmented colored liquids from four images. The ratio between the colored area and the entire implant projection area is used to estimate the average coverage ratio. FIG. 5C displays the average coverage ratios of liquids for three distinct gap size ratios: δr/r=0.01, 0.05, and 0.1. The average coverage ratio β is defined as the projected wetted area Al over the entire projected area of the dental implant As. Furthermore, FIG. 5D illustrates the variance of ejected liquid in terms of covered area, indicating the spread of liquid distribution. This variance is defined as the diameter of the long axis of an ellipse fitted over the wetted area. Additionally, FIG. 5E presents the liquid ejection processes for different gap sizes. For smaller gap sizes, only a limited amount of liquid is expelled, primarily within the grooved pattern, resulting in a small coverage ratio and variance. As the gap sizes increase, the liquid extends to wet the screw pattern more comprehensively and distributes more evenly. However, with a further increase in the gap size ratio to 0.1, the liquid coverage ratio becomes limited to a portion of the phantom due to the restricted liquid volume, and the variance of the liquid distribution is also diminished.

The circular pressure from the surrounding material may prevent the liquid ejection and spreading. The depth of the groove pattern on the outer surface of the dental screw should optimize the liquid coverage ratio and distribution. As illustrated in FIG. 5F, with a groove width of 0.4 mm, the groove depth, denoted as dg, is varied from 0.13 to 0.63 mm, achieved through 3D printing techniques. FIGS. 5G and 5H show the impact of groove depth on liquid coverage ratio and variance. When the groove is shallow, effective ejection of liquid from the main chamber becomes challenging due to circular pressure. Conversely, as the groove depth increases, the coverage area on the implant surface diminishes. For instance, with a groove depth of 0.63 mm as shown in FIG. 5I, liquid ejection within a phantom featuring a gap size of δr/r=0.05 is demonstrated. Consequently, it is suggested to select a groove depth within the moderate range (≈0.43 mm) to optimize the coverage ratio and the distribution.

### Example 5: Demonstration of Liquid Election Inside Porcine Bones Using a Metal Dental Implant

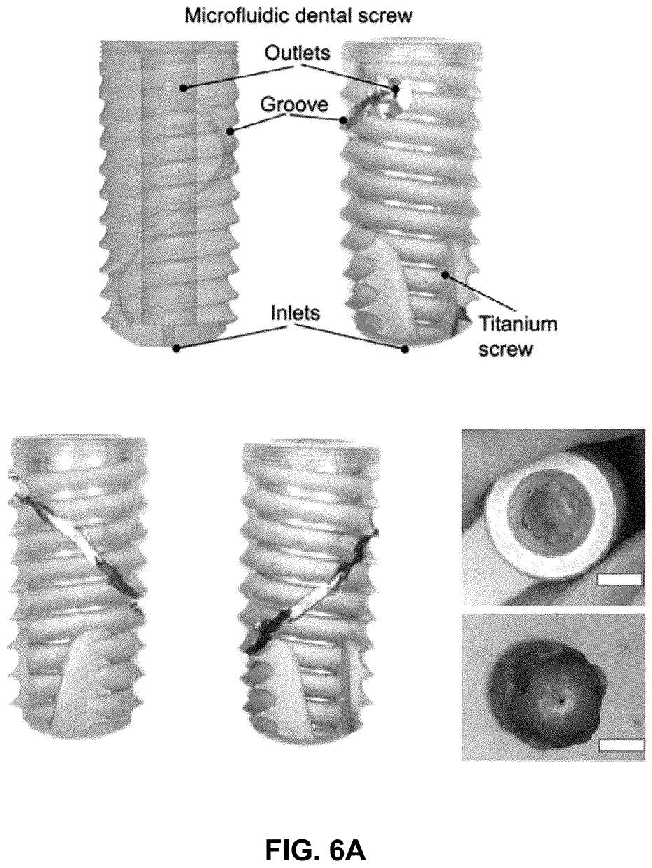

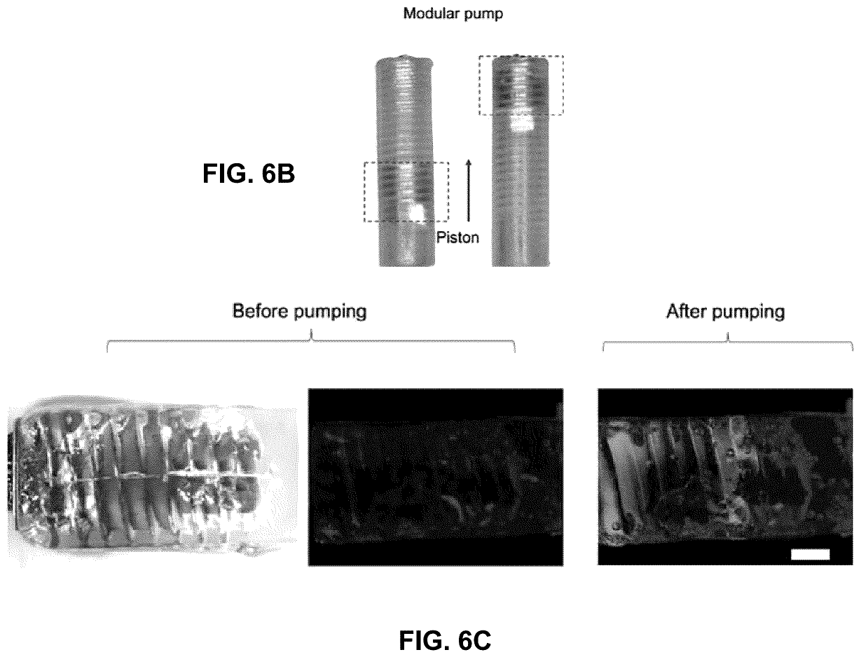

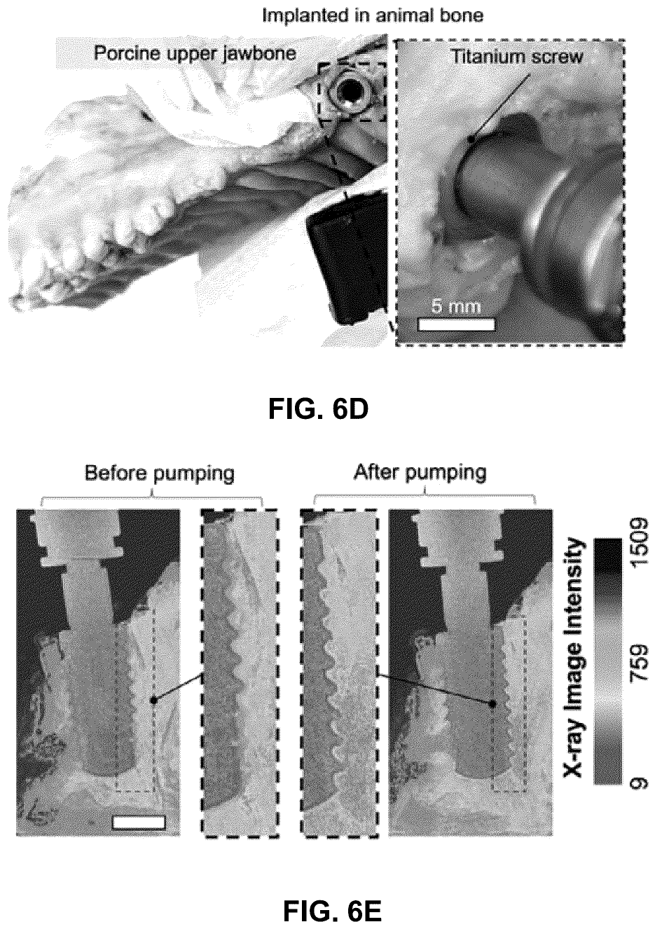

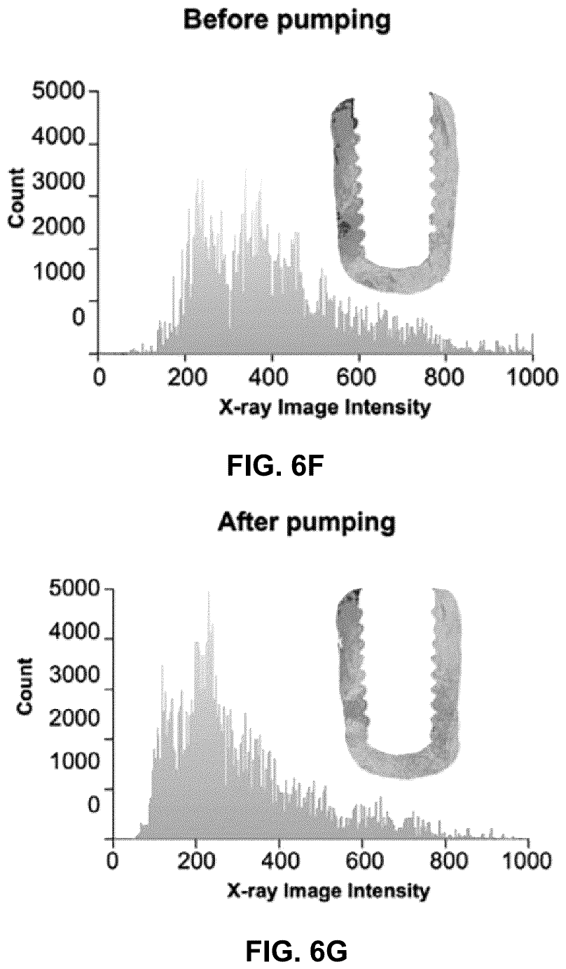

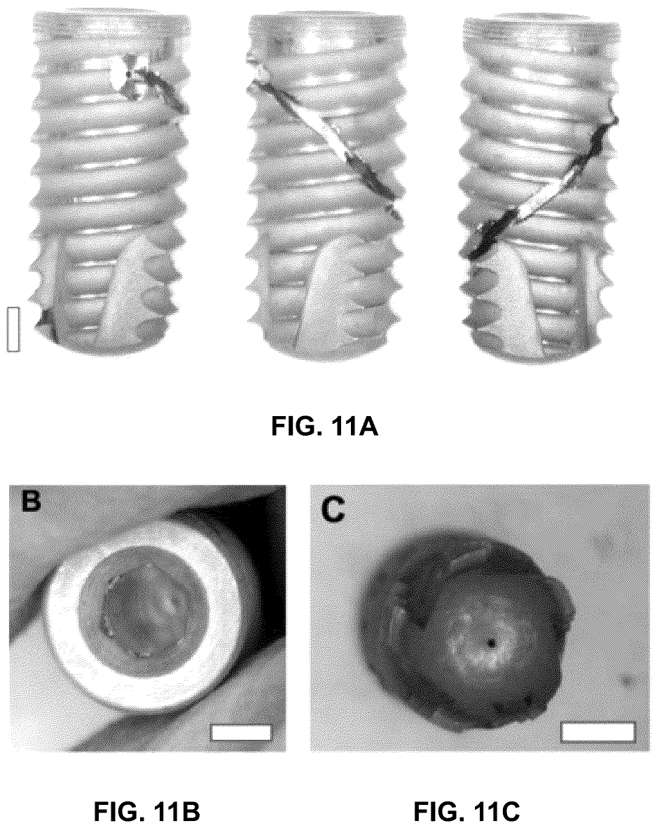

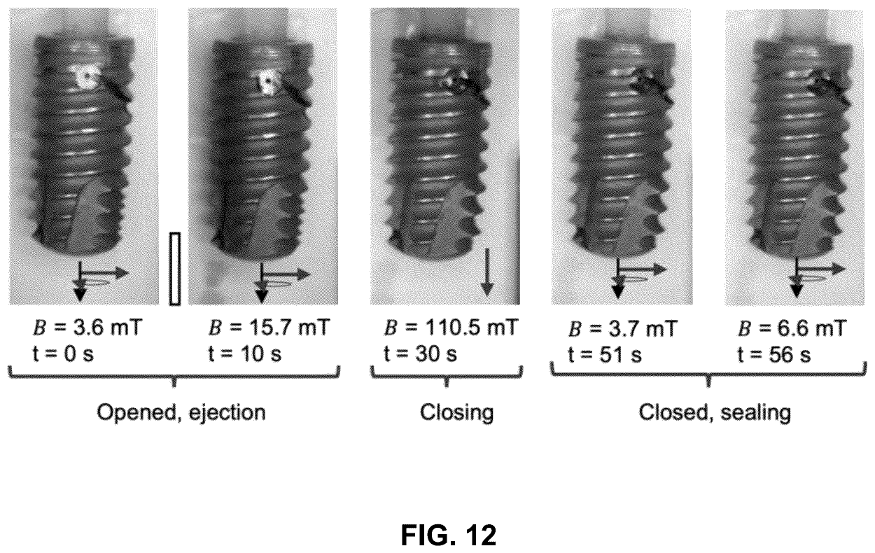

To explore the efficacy of liquid release mechanisms within a porcine maxilla (jawbone), the present disclosure fabricated a metal dental screw featuring micromachined inner channels and outer groove patterns, integrating modular pumps and valves within the dental implant structure. This metal dental screw is crafted by modifying a titanium implant (6 mm in diameter, 13 mm in length, Cyclone Implant Tapered Self Drilling Dental Implant—Internal Hex, by Bhi Implants Ltd.) through a metal machining processes (FIGS. 8A-8B). Using an EDM drilling machine (AgieCharmillesDRILL 20, +GF+), three holes were precisely drilled: an M3 through hole, and two 0.2 mm through holes, as shown in FIGS. 6A and 11A-11C. A segment of the threaded part is retained to allow mounting of the abutment using a connecting screw. In addition, FIG. 6B shows that the modular pump comprises a resin tube with a diameter of 3 mm and a length of 10 mm, tapped using an M2 (pitch: 0.4 mm) tapping tool. The piston is composed of an M2 by 2 mm set screw bonded with a 1 mm by 1 mm by 1 mm magnet (NdFeB, N45). In the context of the metal implant, FIG. 6C shows the process of embedding the metal dental implant within a PDMS dental phantom. Activation of the magnetic valve occurs upon application of a 110 mT magnetic field, while a rotating magnetic field of 15 mT and 0.1 Hz facilitates liquid ejection. Notably titanium's weak magnetic properties do not interfere with the motion of the magnetic piston. By t=1.5 min, the presence of fluorescent dye, as shown in FIG. 6C, serves as evidence of the efficacy of the metal dental implant featuring the integrated pump and valve system. Meanwhile, FIG. 12 also shows the sealing ability of the magnetic valve to prevent leaking of the liquids and allow liquid to be ejected out when being locked open.

To demonstrate the feasibility of the implant inside an animal bone, the dental implant was implanted within a porcine maxilla (jawbone) to observe liquid ejection. As illustrated in FIG. 6D, the metal implant is surgically inserted into the maxilla using a dental tool set (Universal Prosthetic Torque Wrench Screwdriver Tool, BUIFAC) following standard dental implant placement procedures. Subsequently, the dental implant is filled with a combination of liquid and contrast agent (Omnipaque iohexol injection, GE Healthcare). Following the implantation, X-ray images are collected in an X-ray cabinet imaging machine (Faxitron MX-20 Specimen Radiography System, Faxitron X-ray, LLC) to compare the change of X-ray intensity around the dental implant. The peri-implant X-ray intensity contours before and after pumping contrast agent are shown in FIG. 6E. A discernible decrease of the X-ray intensity was observed around the dental implant after pumping. The peri-implant X-ray intensity histograms in FIGS. 6F and 6G further indicate a shift from high X-ray image intensity distribution before pumping (FIG. 6F) to a low X-ray image intensity distribution of the contrast agent after pumping (FIG. 6G) in the segmented peri-implant region. The comparison indicates the contrast agent release from the dental implant to the porcine maxilla in this proof-of-concept demonstration.

### Example 6: Additional Discussion Relating to Examples 1-5

In summary, the present disclosure presents a wirelessly actuated pump and valve in dental implants for liquid cargo ejection. The present disclosure has showcased the pumping and valve control functions in a dental implant, which could be actuated by a magnetic field to allow on-demand and controllable liquid release on the implant surface. The components of the device, including the magnetic valve and piston pump, have been shown to be wirelessly actuated with a simple hand-hold magnet. To alleviate the circular pressure from the maxilla, the dental screw has a grooved pattern to allow liquid to flow to the implant surface when needed. The effect of the groove pattern depth on the wetting ability has also been investigated. The efficiency of the magnetic valve control has been validated, which could effectively avoid accidental release of the drug. Antibacterial tests were performed in vitro in a phantom model. Finally, the ability of ejecting liquids inside an animal jawbone has been validated in a proof-of-concept ex vivo study.

Compared with other wirelessly controlled drug releasing methods such as remote heating by RF magnetic fields,[26] the present disclosure takes advantage of the magnetic field induced mechanical pressure of a screw piston to release liquids with a larger penetration depth and a smaller footprint to be integrated in dental implants. The presently disclosed wirelessly actuated pump and valve mechanisms hold potential for delivering antibiotics to prevent biofilm formation[40] while conventional dental implants are at risk for peri-implant mucositis when periodontal pathogens grow around the surface of the implant.[41,42] The system could be used for delivering therapeutic stem cells in bone implants for enhanced osseointegration. Moreover, the presently disclosed dental implant may be engineered to allow refilling by incorporating a small inlet, enabling the drug chamber to be replenished for long-term applications. Overall, by leveraging magnetic fields for precise control and targeted drug delivery, the present disclosure opens new possibilities for improving patient outcomes and enhancing the effectiveness of implant-based therapies.

### Example 7: Additional Details to Relating Examples 1-6

Fabrication of the Modular Piston Pump: The modular pump for the dental implant was printed with UV-curable resin (Clear resin V4, Formlabs, Inc) in a stereo-photolithography (SLA) 3D printer (Form 3+, Formlabs, Inc.). After printing, the printed parts were cleaned in isopropyl alcohol in an ultrasonic cleaner for 20 min to remove the uncured liquid resin. The cleaned parts were exposed to UV light (wavelength: 365 nm) for secondary cure for ≈30 min at a temperature of 65° C. The cylindrical tubes were tapped using an M2 (pitch: 0.4 mm) tapping tool. The piston comprises an M2 by 2 mm set screw made of steel bonded with a 1 mm by 1 mm by 1 mm NdFeB magnet (Degrade, N45; SuperMagnetMan). The magnet was aligned with the screw such that the magnetic axis was perpendicular to the screw. The connecting surfaces of the magnet and screw were roughened, and super glue was applied at the interface and the two were pressed together.

Fabrication of the Lockable Valve: The lockable valve was fabricated by assembling a foldable magnetic leaf (Ecoflex 00-30 and NdFeB), base layer (PDMS), and the locking mechanism (PDMS). First, poly(methyl methacrylate) (PMMA) substrates were prepared with a 140 μm thick spacer using two layers of PET tapes. PDMS with a weight ratio of 10:1 between the monomer and the cross-linker was poured onto the PMMA substrate, scraped with razor blades, and cured in an oven for 15 min at 120° C. Subsequently, the PDMS was cut into modules of different designs using a laser machine (LPKF ProtoLaser U4, LPKF Laser & Electronics North America). Second, the valve body was prepared with the PDMS modules. The base layer is a disk with an outer diameter of 1.9 mm and a cut-out hole of 0.8 mm in diameter. Meanwhile, the locking mechanism was made by assembling spacers (controlling height) and stoppers (locking the magnetic leaf). The spacers are in an arch-shape with an outer diameter of 1.9 mm, an inner diameter of 1.5 mm, and a width of 0.4 mm. The stopper was made of arch-shaped segments with an outer diameter of 1.9 mm and height of 0.5 mm. The plug is a cylinder made of PDMS with a diameter 0.8 mm and height of 280 μm. Third, to make the flexible magnetic leaf, Ecoflex 00-30 silicone rubber (Smooth-On, Inc.) and NdFeB magnetic particles (MQFP-15-7, average diameter, 5 μm; Magnequench International, LLC.) were mixed (weight ratio, 1:2) and poured onto a glass substrate with a 140 μm thick spacer. After being scraped and cured in an oven at 90° C. for 20 min, the magnetic composite was cut into a disk shape with an inner diameter of 1.4 mm and outer diameter of 1.9 mm. The leaf was then magnetized in an impulse magnetizer (IM-10-30, ASC Scientific) with a 1.8 T magnetic field impulse along the body-axis. The previously prepared plug was bonded to the center of the magnetic disk using liquid PDMS (10:1 by weight). Finally, the magnetic leaf and the locking mechanism were bonded on the base layer using uncured PDMS. The resulting valve was cured in an oven at 120° C. for 5 min.

Fabrication of the Resin Dental Screw with Holes and Grooves: The resin dental screw was printed with UV-curable resin (Clear resin V4, Formlabs, Inc) in a stereo-photolithography (SLA) 3D printer (Form 3+, Formlabs, Inc.) based on a 3D design prepared in Solidworks 2022 (Dassault Systemes). After printing, the molds were cleaned in isopropyl alcohol in an ultrasonic cleaner for 20 min until all excess resin was removed. Subsequently, the molds were exposed to UV light (wavelength: 365 nm) and heated to 65° C. to cure for 30 min. In addition, a two-step transfer molding method was also used to mold dental screws with non-UV resins (Smooth-Cast 322, Smooth-On Inc.) when testing with fluorescent dyes. For both the 3D printed and molded dental screws, a 0.4 mm drill (Shank Tungsten Carbide Micro Drill Bits Set, Lothee Inc.) was used to create an inlet, and a 0.5 mm drill was used to create the outlet. The implant was subsequently tapped using an M2 by 0.4 mm tapping tool. For the fluorescence imaging experiments, the dental screw was further casted using non-UV curable resin by transfer molding method. A typical set of parameters for the 3D-printed or molded dental implants and microfluidic units are shown in the table in Example 9.

Fabrication of the Metal Dental Screw with Holes and Grooves: The metal dental screw was prepared by machining fluidic channels on a titanium implant (Cyclone Implant Tapered Self Drilling Dental Implant—Internal Hex, Bhi Implants Ltd.) by metal machining. An M3 through hole and two 0.2 mm through holes were all drilled using an EDM drilling machine (AgieCharmilles DRILL 20, +GF+). Computer numerical control machine (GENOS M460-VE, Okuma America Corporation) was used to further milling the grooves on the outer surface of the dental screw. The optical images of the machined dental screw are shown in FIGS. 11A-11C. To further allow biocompatibility, PDMS coating was first applied, and then parylene-C coating was applied using a parylene deposition system (PDS 2010, SCS Labcoter 3, Specialty Coating Systems).

Preparation of the Tooth Phantom and Animal Bone: The testing phantoms were made by molding. First, positive molds of dental screws with different sizes were printed with UV-curable resin (Clear resin V4, Formlabs Inc.) in a resin 3D printer (Form 3+, Formlabs Inc.). Then, containers with dimensions of 8.5 mm by 8.5 mm by 18 mm were 3D printed to serve as mold holders. After printing, the molds and containers were cleaned in isopropyl alcohol in an ultrasonic cleaner for 20 min. Subsequently, the printed dental screws and containers were exposed to UV (365 nm) and heated at 65° C. to cure. They were then coated with Trichloro(1H,1H,2H,2H-perfluorooctyl)silane (97%, Sigma-Aldrich, Inc.) using vacuum deposition to facilitate curing PDMS. The container was fully filled with PDMS (Dow Silicones Corporation) with a weight ratio of 5:1 between the monomer to the cross-linker and then the dental screws of different sizes were placed inside the containers with subsequent degassing for 10 min in a vacuum chamber to remove air bubbles. A negative mold was demolded as a dental phantom after curing the sample in an oven at 90° C. for 80 min. In addition, the porcine upper jawbone was purchased from a local slaughterhouse close to Houston, Texas. The bone was cut and sequentially drilled with increasingly large diameter drills (Universal Prosthetic Torque Wrench Screwdriver Tool, BUIFAC) and the dental implant was then screwed into the osteotomy.

Procedure for Testing Ejection of Liquid with Fluorescent Dyes in a Phantom: To perform the liquid ejection testing, first, the piston was inserted into the bottom of the tested dental screw by a rotating magnetic field. Then, oil was dipped a little bit on the top of piston to avoid fluorescent dyes leaking down through the screw. After adding oil, the dental screw was filled with fluorescent dyes and sealed with a block. Finally, the dental screw was inserted into the tested phantom by rotating with the thread line. The experimental arrangement for assessing liquid ejection with fluorescent dyes in a phantom comprised several components: a black chamber housing three 365 nm UV lamps, a USB camera, a table jack, a stepper motor, a motor driver, and an embedded controller (Arduino Uno). The USB camera and prepared phantom were situated on the table jack, while the motor, equipped with a permanent magnet, was positioned ≈4 cm away from the phantom. The table jack's height was adjusted to ensure horizontal alignment between the magnet and the phantom. The stepper motor, controlled by the embedded controller, operated at a speed of 30 RPM. The entire process of liquid ejection was conducted within the black chamber illuminated by UV lamps, with the USB camera recording the ejection process. Following liquid ejection, the phantom was systematically rotated by 90° to display all four surfaces. Each experimental group was repeated five times for consistency. Recorded videos were initially processed using MATLAB to segment the colored liquids and determine the occupied pixel count, representing the covering area. Subsequently, the total area of the dental screw phantom was computed by outlining a polygon that matched its shape. The coverage ratio was then calculated by dividing the total pixel count of colored liquids by the total pixel count of the dental screw phantom. Additionally, the colored region was fitted with an ellipse, enabling the calculation of the ellipse's major axis.

Procedure for Testing Ejection of Liquid with Contrast Agent in an Animal Bone: The porcine upper jawbone was procured from a local slaughterhouse near Houston, Texas, and carefully preserved in a freezer. Prior to the commencement of the experiment, the jawbone underwent a defrosting process and was meticulously drilled to create a 5 mm aperture, utilizing a specialized dental tool set. Subsequently, the dental implant was infused with a contrast agent (Omnipaque iohexol injection, GE Healthcare) before being securely affixed into the predrilled osteotomy. To accommodate the experimental setup, the jawbone was trimmed to ≈50 mm in length, ensuring proper fit within a square plastic container measuring 50 by 50 by 50 mm. This container was then positioned within an X-ray medical imaging cabinet (Faxitron MX-20 Specimen Radiography System). Before initiating the magnetic piston pump, sequential X-ray images were captured at intervals of 90°, achieved through the rotation of the plastic container. The rotation mechanism was facilitated by a permanent magnet affixed to a stepper motor, ensuring precise and controlled movement of the piston. Upon activation of the magnetic piston pump, the contrast agent was expelled from the implant, facilitating visualization within the jawbone. Following this injection, additional X-ray images were acquired from the same angles within the cabinet system, providing comprehensive documentation of the experimental process.

Magnetic Actuation Systems: A 25 mm by 25 mm by 25 mm NdFeB magnet was mounted on a step motor controlled by a step motor driver (L298N, HiLetgo) for testing the liquid ejection in the dental implants. For the handheld system, a 10 mm by 10 mm by 10 mm cube magnet was mounted on a DC motor and further driven by a 2-Channel H-Bridge DC motor driver. An Arduino Nano Every embedded controller was used for controlling the speed of the DC motor driver. The speed was controlled using a rotational encoder.

Analysis of the Contrast Agent Distribution in the X-Ray Images: Image registration was performed on X-ray images before and after pumping to digitally overlap the dental implant in the images. The images were then processed using the delineate function in Avizo software (Thermo Fisher Scientific, Burlington, MA) to visually enhance the implant boundary. The X-ray intensity distribution in peri-implant region was extracted from Avizo as histograms.

Statistical Analysis: Data presentation (mean and standard deviations) and sample size (n) for each statistical analysis were mentioned in the figure caption. In FIGS. 5C, 5D, 5G, and 5H, liquid ejections of n=5 trials were performed for each dental screw inside a specific phantom. The circles indicate the average values for the coverage ratios or the variance of the fitted ellipse of n=20 segmented images in total for the four faces in five trials. MATLAB 2023a (Mathworks Inc.) was used for the statistical analysis.

### Example 8: Additional Details

In an embodiment, the dimensions of dental implants as described herein can be the following values, or about the following values:

| Parameter | Name | Value | |
|---|---|---|---|
| w_{g} | Groove width | 0.4 | mm |
| d_{g} | Groove depth | 0.43 | mm |
| Dₕᵢ | Inlet hole diameter | 0.4 | mm |
| Dₕₒ | Outlet hole diameter | 0.6 | mm |
| Dₘᵢ | Modular pump inner diameter | 2.7 | mm |
| Dₘₒ | Modular pump outer diameter | 3 | mm |
| Lₚₘ | Piston cube magnet length | 1 | mm |
| Dₚₛ | Piston screw diameter | 1.9 | mm |
| Lₚₛ | Piston screw length | 2 | mm |
| Dᵥ | Valve diameter | 1.8 | mm |
| hᵥₛ | Height of the valve stopper | 0.42 | mm |

### Example 9: Additional Details

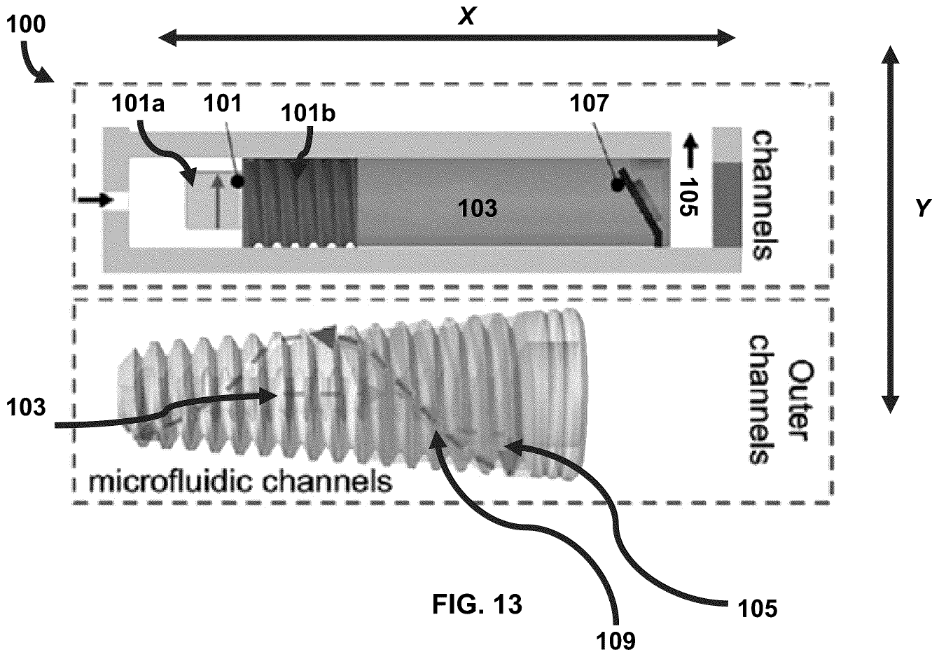

FIG. 13 shows additional aspects of an embodiment of an implant 100 according to the present disclosure. As can be seen in FIG. 13, the implant 100 comprises a magnetic screw piston 101 (i.e., actuator) with a magnet 101a bonded to a screw 101b. An axial internal fluidic chamber 103 comprises a fluid that can be ejected from the device upon actuation of the magnetic screw piston 101 (axial axis X). Upon actuation of the magnetic screw piston 101, the fluid in the axial internal fluidic channel 103 can be ejected through the radial channel 105 upon opening of the magnetic leaf valve 107 (radial axis Y). The fluid then flows through the radial channel 105 to the outer channel 109 (i.e., outer microfluidic channel comprising a helical groove transversing outer threads) and into the subject. Addition details of the magnet screw piston (i.e., actuator) and magnetic valve (also referred to as “magnetic leaf” are shown, for example, in FIGS. 2B-2D.

### REFERENCES RELATED TO THE PRESENT DISCLOSURE, IN PARTICULAR, EXAMPLES 1-9

[1] Y. Zhang, F. Liu, Y. Zhang, J. Wang, D. D'Andrea, J. B. Walters, S. Li, H.-J. Yoon, M. Wu, S. Li, Z. Hu, T. Wang, J. Choi, K. Bailey, E. Dempsey, K. Zhao, A. Lantsova, Y. Bouricha, I. Huang, H. Guo, X. Ni, Y. Wu, G. Lee, F. Jiang, Y. Huang, C. K. Franz, J. A. Rogers, Proc. Natl. Acad. Sci. USA 2023, 120, 2217734120.
[2] J. Koo, S. B. Kim, Y. S. Choi, Z. Xie, A. J. Bandodkar, J. Khalifeh, Y. Yan, H. Kim, M. K. Pezhouh, K. Doty, G. Lee, Y.-Y. Chen, S. M. Lee, D. D'Andrea, K. Jung, K. Lee, K. Li, S. Jo, H. Wang, J.-H. Kim, J. Kim, S.-G. Choi, W. J. Jang, Y. S. Oh, I. Park, S. S. Kwak, J.-H. Park, D. Hong, X. Feng, C.-H. Lee, et al., Sci. Adv. 2020, 6, 20.
[3] F. P. Pons-Faudoa, A. Ballerini, J. Sakamoto, A. Grattoni, Biomed. Microdevices 2019, 21, 47.
[4] S. M. Hayek, T. L. Yaksh, A. D. Sharan, Neuraxial Therapeutics: A Comprehensive Guide, Springer International Publishing, Cham, 2024, pp. 729-753.
[5] K. B. Sutradhar, C. D. Sumi, Drug Delivery 2016, 23, 1.
[6] E. Magill, S. Demartis, E. Gavini, A. D. Permana, R. R. S. Thakur, M. F. Adrianto, D. Waite, K. Glover, C. J. Picco, A. Korelidou, U. Detamomrat, L. K. Vora, L. Li, Q. K. Anjani, R. F. Donnelly, J. Dominguez-Robles, E. Larrañeta, Adv. Drug Delivery Rev. 2023, 199, 114950.
[7] I. C. F. Pereira, R. J. S. van Mechelen, H. M. Wyss, L. Pinchuk, H. J. M. Beckers, J. M. J. den Toonder, Microsyst. Nanoeng. 2023, 9, 92.
[8] S. M. Won, L. Cai, P. Gutruf, J. A. Rogers, Nat. Biomed. Eng. 2021, 7, 405.
[9] E. C. Wisdom, Y. Zhou, C. Chen, C. Tamerler, M. L. Snead, ACS Biomater. Sci. Eng. 2020, 6, 2682.
[10] M. D. Soler, S. M. Hsu, C. Fares, F. Ren, R. J. Jenkins, L. Gonzaga, A. E. Clark, E. O'neill, D. Neal, J. F. Esquivel-Upshaw, Materials 2020, 13, 5488.
[11] D. F. Kinane, P. G. Stathopoulou, P. N. Papapanou, Nat. Rev. Dis. Primers 2017, 3, 17038.
[12] J. Hasan, R. Bright, A. Hayles, D. Palms, P. Zilm, D. Barker, K. Vasilev, ACS Biomater. Sci. Eng. 2022, 8, 4697.
[13] K. Gulati, C. Ding, T. Guo, H. Guo, H. Yu, Y. Liu, Int. J. Oral Sci. 2023, 15, 15.
[14] G. M. Esteves, J. Esteves, M. Resende, L. Mendes, A. S. Azevedo, Antibiotics 2022, 11, 235.
[15] S. Kligman, Z. Ren, C. H. Chung, M. A. Perillo, Y. C. Chang, H. Koo, Z. Zheng, C. Li, J. Clin. Med. 2021, 10, 1641.
[16] A. M. Inchingolo, G. Malcangi, L. Ferrante, G. Del Vecchio, F. Viapiano, A. D. Inchingolo, A. Mancini, C. Annicchiarico, F. Inchingolo, G. Dipalma, E. Minetti, A. Palermo, A. Patano, J. Funct. Biomater. 2023, 14, 287.
[17] S. A. Stewart, J. Dominguez-Robles, R. F. Donnelly, E. Larrañeta, Polymers 2018, 10, 1379.
[18] D. R. Bijukumar, C. McGeehan, M. T. Mathew, Curr. Osteoporosis Rep. 2018, 16, 236.
[19] M. Yu, D. You, J. Zhuang, S. Lin, L. Dong, S. Weng, B. Zhang, K. Cheng, W. Weng, H. Wang, ACS Appl. Mater. Interfaces 2017, 9, 19698.
[20] S. Wu, J. Xu, L. Zou, S. Luo, R. Yao, B. Zheng, G. Liang, D. Wu, Y. Li, Nat. Commun. 2021, 12, 3303.
[21] J. Li, D. J. Mooney, Nat. Rev. Mater. 2016, 1, 16071.
[22] H. K. Kim, Y. S. Park, Drug Delivery 2017, 24, 437.
[23] S. Xu, Y. Chen, N. S. P. Hyun, K. P. Becker, R. J. Wood, Proc. Natl. Acad. Sci. USA 2021, 118, 2103198118.
[24] H. Lin, J. Tan, J. Zhu, S. Lin, Y. Zhao, W. Yu, H. Hojaiji, B. Wang, S. Yang, X. Cheng, Z. Wang, Nat. Commun. 2020, 11, 4405.
[25] J. Zhang, J. Xu, J. Lim, J. K. Nolan, H. Lee, C. H. Lee, Adv. Healthcare Mater. 2021, 10, 2100194.
[26] H. R. Cheong, N. T. Nguyen, M. K. Khaw, B. Y. Teoh, P. S. Chee, Lab Chip 2018, 18, 3207.
[27] B. J. Nelson, S. Gervasoni, P. W. Y. Chiu, L. Zhang, A. Zemmar, Proc. IEEE 2022, 110, 1028.
[28] Y. Dong, L. Wang, V. Iacovacci, X. Wang, L. Zhang, B. J. Nelson, Matter 2022, 5, 77.
[29] B. Xiao, Y. Xu, S. Edwards, L. Balakumar, X. Dong, Adv. Funct. Mater. 2024, 34, 2307751.
[30] Y. Xu, B. Xiao, L. Balakumar, K. L. Obstein, X. Dong, IEEE Rob. Autom. Left. 2023, 8, 5720.
[31] G. Z. Lum, Z. Ye, X. Dong, H. Marvi, O. Erin, W. Hu, M. Sitti, Proc. Natl. Acad. Sci. USA 2016, 113, 6007.
[32] K. Kordbacheh Changi, J. Finkelstein, P. N. Papapanou, Clin. Oral Implants Res. 2019, 30, 306.
[33] M. Wada, T. Mameno, M. Otsuki, M. Kani, Y. Tsujioka, K. Ikebe, Jpn. Dent. Sci. Rev. 2021, 57, 78.
[34] I. L. C. Chapple, R. Genco, J. Periodontol. 2013, 84, S106.
[35] M. Chmielewski, A. Pilloni, Dent. J. 2023, 11, 134.
[36] V. Madiwal, B. Khaimar, J. Rajwade, iScience 2024, 27, 108827.
[37] Z. Shi, Y. Lu, S. Shen, Y. Xu, C. Shu, Y. Wu, J. Lv, X. Li, Z. Yan, Z. An, C. Dai, npj Flexible Electron. 2022, 6, 49.
[38] M. Veletic, E. H. Apu, M. Simic, J. Bergsland, I. Balasingham, C. H. Contag, N. Ashammakhi, Chem. Rev. 2022, 122, 16329.
[39] M. Veletic, E. H. Apu, M. Simic, J. Bergsland, I. Balasingham, C. H. Contag, N. Ashammakhi, Chem. Rev. 2022, 1, 16329.
[40] G. Hwang, A. J. Paula, E. E. Hunter, Y. Liu, A. Babeer, B. Karabucak, K. Stebe, V. Kumar, E. Steager, H. Koo, Sci. Rob. 2019, 4, 2388.
[41] J. Liang, X. Peng, X. Zhou, J. Zou, L. Cheng, Molecules 2020, 25, 516.
[42] M. Chmielewski, A. Pilloni, Dent. J. 2023, 11, 134.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1) A dental implant comprising:
an internal fluidic chamber extending axially through the implant;
one or more inner microfluidic channels extending radially from the internal fluidic chamber to one or more outer microfluidic channels;
one or more outer microfluidic channels, wherein the outer microfluidic channels comprise a helical groove transversing outer threads of the implant;
an actuator configured within the internal fluidic chamber to pump liquid through the implant in the presence of a magnetic field;
a flexible magnetic valve configured between the internal fluidic chamber and the one or more inner microfluidic channels, wherein the flexible magnetic valve is opened or closed through application of an axial external magnetic field; and
a stopper structure bonded to the flexible magnetic valve, wherein the stopper structure maintains an opening angle or a closed position of the flexible magnetic valve after the axial external magnetic field has been removed.

2) The implant of claim 1, wherein the actuator comprises a magnet-screw piston.

3) The implant of claim 1, wherein the actuator comprises:
a screw bonded to a magnet, wherein an epoxy bonds the screw to the magnet; and
a biocompatible coating.

4) The implant of claim 3, wherein the screw comprises stainless steel, titanium, or both.

5) The implant of claim 3, wherein the magnet is a NdFeB magnet.

6) The implant of claim 3, wherein the coating comprises PDMS, parylene-C, or both.

7) The implant of claim 1, wherein the stopper structure is coated with parylene-C.

8) The implant of claim 1, wherein the internal fluidic chamber comprises a small inlet, wherein the internal fluidic chamber can be refilled with the liquid.

9) The implant of claim 1, wherein the implant comprises titanium.

10) The implant of claim 1, wherein the flexible magnetic valve is bonded to the stopper structure using uncured PDMS.

11) A system for delivering a liquid cargo within a dental implant, comprising:
the dental implant of claim 1; and
a magnetic actuation unit, further comprising
a magnet, wherein the magnet is mounted onto a step motor;
a step motor, wherein the step motor is controlled by a step motor driver;
a battery, wherein the battery is connected to the step motor driver and a microcontroller, wherein the microcontroller controls the speed of the step motor driver; and
a switch, wherein the switch is connected to the microcontroller and allows a user to modify the speed of the step motor driver;
wherein, the magnetic actuation unit generates the external magnetic field, controlling delivery of the liquid through the implant.

12) The system of claim 11, wherein the actuator comprises:
a stainless steel or titanium screw bonded to a NdFeB magnet, wherein an epoxy bonds the screw to the magnet; and
a biocompatible coating, wherein the coating further comprises PDMS, parylene-C, or both.

13) The system of claim 11, wherein the stopper structure is coated with parylene-C.

14) The system of claim 11, wherein the internal fluidic chamber comprises a small inlet, wherein the internal fluidic chamber can be refilled with the liquid.

15) The system of claim 11, wherein the flexible magnetic valve is bonded to the stopper structure using uncured PDMS.

16) A method of introducing a medicament to an implant-bone interface, comprising:
implanting a dental implant of claim 1 into the jaw bone of a patient, wherein the implant contains a medicament;
opening a flexible magnetic valve within the implant, wherein the flexible magnetic valve is opened by application of an axial external magnetic field;
activating an actuator within the implant, wherein the actuator pumps the medicament through the flexible magnetic valve and out of the implant by application of a rotating external magnetic field; and
delivering the medicament from the implant directly to the implant-bone interface.

17) The method of claim 16, further comprising:
refilling the implant with the medicament, wherein the implant is refilled by injecting the medicament into a small inlet of the implant.

18) The method of claim 17, wherein the medicament comprises antibiotics, stem cells, coatings, small molecules less than 2500 daltons, or a combination of any thereof.

19) The implant of claim 16, wherein the actuator is further configured to translate along the axis of the internal fluidic chamber when the rotating magnetic field is applied.

20) The system of claim 16, wherein the actuator is further configured to translate along the axis of the internal fluidic chamber when the rotating magnetic field is applied by the magnetic actuation unit.
